SEQUENCE LISTING (6 sequences)

SEQ ID NO: 1 - protein, 18 aa, Artificial Sequence
  Description of Artificial Sequence Synthetic Peptide
MLCCMRRTKQVEKNDDQK

SEQ ID NO: 2 - DNA, 17 bp, Mus musculus
gtcccaggtcccgaaaa

SEQ ID NO: 3 - DNA, 68 bp, Artificial Sequence
  Description of Artificial Sequence Consensus splice donor site
ccgctcgagacttacctgactggccgtcgttttaagacgagctccctagctagtcaggcaccgggctt

SEQ ID NO: 4 - protein, 6 aa, Artificial Sequence
  Description of Artificial Sequence Consensus motif
MGXXXX

SEQ ID NO: 5 - protein, 4 aa, Artificial Sequence
  Description of Artificial Sequence Illustrative CRD motif
DHHC

SEQ ID NO: 6 - protein, 13 aa, Artificial Sequence
  Description of Artificial Sequence C-term tail peptide of GFP Yck2p
KSSKGFFSKLGCC

(19) United States
(12) Patent Application Publication
(10) Pub. No.: US 2008/0206744 A1
(43) Pub. Date: Aug. 28, 2008

(54) Functional Genomics and Gene Trapping in Haploid or Hypodiploid Cells

(76) Inventor: David Alan Zacharias, Palm Coast, FL (US)

Correspondence Address: FOLEY & LARDNER LLP, P.O. BOX 80278, SAN DIEGO, CA, 92138-0278, US

(21) Appl. No.: 10/590,633

(22) PCT Filed: Feb. 25, 2005
(86) PCT No.: PCT/US05/06309

§ 371 Date: Jul. 6, 2007

Related U.S. Application Data
(60) Provisional application No. 60/548,509, filed on Feb. 26, 2004.

(51) Int. Cl. C12Q 1/68; C12N 5/00

(52) U.S. Cl. 435/6; 435/325

(57) ABSTRACT

The present invention provides methods and compositions for performing functional genomics and gene trapping using haploid cells, including haploid or hypodiploid vertebrate cells. The present invention further provides methods for identifying genes involved in cellular signaling pathways.

### RELATED APPLICATIONS

This application claims benefit of provisional patent application no. 60/548,509, filed Feb. 6, 2004, the entire contents of which are hereby incorporated by reference herein.

### FIELD OF THE INVENTION

The present invention relates to methods and compositions for performing functional genomics and gene trapping using haploid or hypodiploid cells, including haploid or hypodiploid vertebrate cells, in combination with high throughput imaging. In a particular aspect, the present invention relates to methods for identifying genes involved in cellular signaling pathways.

### BACKGROUND OF THE INVENTION

Gene trapping or random insertional mutagenesis is a method used to discover genes responsible for a particular phenotypic characteristic of an organism. Traditionally a mutagenic element, sometimes also containing a reporter element, is introduced in a stochastic and random way into the genome of embryonic stem (ES) cells by means of a viral vector and/or electroporation. The randomly-mutagenized ES cell lines are characterized and then possibly selected on the basis of some morphological, biochemical or other criterion, then injected into blastocysts, which are implanted into females and go on to form chimaeric animals. Animal lines harboring the mutation of interest in the germline tissue are then bred to homozygosity and the resulting phenotype studied in the whole, mutant animal, or in some tissue or cell of interest taken from the mutant animal.

The process of generating mutant animals in this fashion is very time consuming, as well as being labor and cost intensive to the point of being prohibitive for many research facilities. Likewise, the time involved is also substantial requiring many months before experiments on the “gene-trapped” animal can begin. The motivation to use such a system is that most commonly used cell lines are diploid and as such insertion of a mutagenic element will at most probably affect only one of two alleles of a gene present in the genome. It is unsafe to assume, and indeed very unlikely, that inactivation of a single allele will be sufficient to eliminate totally the function of a particular gene, thereby necessitating elimination of both alleles of a diploid cell line.

Accordingly, there is a need in the art for high-throughput screening methods which allow the use of gene-trapping and functional genomics but do not require the generation of live animals.

### SUMMARY OF THE INVENTION

In accordance with the present invention, it has been determined that, if one applies insertional mutagenesis to a cell line that is haploid or hypodiploid, mutation and inactivation of any single gene results in elimination of the function of that gene as there is only a single copy of the gene represented in the haploid or hypodiploid cell line. This is of substantial benefit because performing gene-trapping (e.g., insertional mutagenesis) and functional genomics methods in diploid cells is impractical because of the near-impossibility of knocking out both copies of any particular gene.

Gene trapping in haploid or hypodiploid cells can be used to look at any interesting morphological response which can include such responses as changes in cell size, changes in cell shape, changes in cell number, changes in cell migration, changes in the subcellular distribution or concentration of anything that can be visualized, and the like. Indeed, there are already algorithms commercially available to quantify most any type of morphology which one might choose to study. One of the main differences is in the quality of the algorithms.

As will be readily recognized by those of skill in the art, the utility of the present invention extends to any of the above-referenced applications, morphologies or interests. While a focus of the present specification may be directed to the specific chemical conversion of palmitoylation, this is merely a useful model system for demonstration of the concepts embraced by the invention methods.

The introduction of the mutagenic element into a haploid or hypodiploid cell can occur via a variety of mechanisms, e.g., employing retrovirus or electroporation. For example, a stable haploid cell line exists and is available commercially from ATCC (Accession No. CCL-145). This cell line is fibroblast-like, and adherent, two properties that make it useful for microscopic imaging. These cells provide a genomic composition which facilitates carrying out functional genomics experiments such as random insertional mutagenesis using high-throughput/high content microscopy (HCM).

In accordance with another aspect of the present invention, haploid or hypodiploid lines have been developed from other animals, including mouse and human. Following mutagenesis, cellular morphological or physiological readouts selected to identify specific genes that alter the morphology or physiology of interest can be carried out using HCM.

Other features and advantages of the invention will be apparent from the following detailed description and claims.

### BRIEF DESCRIPTION OF THE FIGURES

FIGS. 1A and 1B depict various lipid modifications. Specifically, FIG. 1A illustrates prenylation, and FIG. 1B illustrates acylation. Each class of modification targets proteins to which they are attached to unique subcellular locales (Melkonian et al., 1999; Moffett et al., 2000; Zacharias et al., 2002). This ability is likely due to their varying chain length, degree of saturation and their physical position on the proteins. Both forms of prenylation occur via stable thioether bonds on the final cysteine of a “CAAX” box at the C-terminus of a protein. Myristoylation occurs via a stable amide bond to the N-terminal glycine of a protein while addition of palmitate occurs most commonly via a labile thioester bond to the side chain of a free, reactive cysteine on the cytoplasmic side of the plasma membrane (PM).

FIGS. 2A-2D provide characterization of the reporter of S-palmitoylation. Thus, FIG. 2A reveals that stably-expressed GAP43:GFP is localized to the PM of MDCK cells, illustrating the remarkable homogeneity in the expression pattern that can be achieved. FIG. 2B reveals that GFP, not fused to any subcellular targeting motif, is expressed throughout the interior of the cell, including the nucleus, illustrating that GFP alone has no inherent targeting signals. FIG. 2C reveals that transiently transfected GAP43:GFP is also expressed on the plasma membrane of cells, except when palmitoylation is inhibited (see FIG. 2D) by pre-incubation of transfected cells in 2-bromopalmitate (2BP) (100 μM) (Webb et al., 2000), illustrating that palmitoylation of the adjacent cysteines on the 18-residue peptide from the N-terminus of GAP43 (NH2-MLCCMRRTKQVEKNDDQK-GFP; SEQ ID NO:1) fused to the N-terminus of GFP is sufficient to retain the protein at the PM (Liu et al., 1993; Arni et al., 1998); there is nothing else inherent in the GAP43 peptide or GFP that will localize this protein to the PM. Cells in FIGS. 2B, 2C and 2D are representative of many having the same morphology.

FIG. 3 demonstrates that the palmitoylation sensor, GAP43:GFP, localizes appropriately to the PM of haploid ICR-2A cells, illustrating with these confocal images that a pathway leading to palmitoylation of the sensor is intact in the cell line. GAP43:GFP is expressed transiently under the control of a CMV promoter in the expression plasmid pcDNA3 (InVitrogen). Scale bar=50 μm.

FIG. 4 illustrates ICR-2A cells expressing GAP43:GFP introduced by pantropic retroviral infection.

FIGS. 5A-5D illustrate the simple case of quantifying whether or not GAP43:GFP is on the PM or in the cytoplasm using the HTM algorithm. In this analysis, MDCK cells (the same images as presented in FIGS. 2A-2D), transiently transfected with GFP alone are presented in FIG. 5A; transiently transfected with GAP43:GFP are presented in FIG. 5B; and transiently transfected with GAP43:GFP in the presence of 100 μM 2BP are presented in FIG. 5C. Using masks that define the plasma membrane and the cytoplasm, the algorithm determined that the PM/cytoplasm ratio was significantly different between PM and cytoplasm localization (see FIG. 5D). The localization of GFP alone (see FIG. 5B) is described by a ratio similar to that of GAP43:GFP under conditions where its localization to the PM has been inhibited by incubation in 100 μM 2BP (see FIG. 5C).

FIG. 6 presents a quantitative analysis of the time course of translocation of GAP43:GFP from the plasma membrane in response to 100 μM 2BP: residence half life of palmitoylation. MDCK cells stably expressing GAP43:GFP were exposed to 100 μM 2BP for 6 hours. During this time, the same field of view of cells was imaged repeatedly in two channels Hoechst/nucleus (see FIG. 6A) and GFP/PM (see FIG. 6B) on the EIDAQ100 (the three images: 6A, 6B and 6C are at time 0). The PM mask (green lines) defined by the program is shown in FIG. 6C; the nucleus is delimited by red lines. By determining the average fluorescence intensity of the area defined by the PM mask (see FIG. 6D), PM labeling was reduced by 80% from the maximum (or starting) density during the 6 hour period. Non-linear least squared fit of the data to a single exponential decay curve (see red line in FIG. 6D) accurately described the data and gave a decay constant, equivalent in this assay to the residence half life of palmitate on this substrate, of 179 minutes or ˜3 hours, the same as other, published estimations for the residence of palmitate on proteins (Lane & Liu, 1997; Wolven et al., 1997).

FIG. 7 presents a schematic representation of the components used for insertional mutagenesis. The structure and arrangement of the components are similar whether electroporating plasmid DNA or infecting by retrovirus except that the retroviral constructs will contain long terminal repeat (LTR) sequences flanking the reporter cDNAs. The following abbreviations are used in this figure:
SA, Splice acceptor (5′-GTCCCAGGTCCCGAAAA-(SEQ ID NO:2) from the mouse engrailed 2 gene);
GFP, Green Fluorescent Protein;
pA, polyadenylation sequence that will follow a series of stop codons (XXX) in all three reading frames;
RSV, a promoter to drive expression of neo, a neomycin resistance gene/protein to enable selection of stable integrants by virtue of their antibiotic resistance;
SD, a consensus splice donor site sequence (5′-CCG CTC GAG ACT TAC CTG ACT GGC CGT CGT TTT AA GAC GAG CTC CCT AGC TAG TCA GGC ACC GGG CTT-(SEQ ID NO:3; see Zambrowicz et al., 1998)) is included to ensure proper splicing with a downstream exon/poly-adenylation site;
Three lines (i.e., |||) represent palmitoyl groups that will localize the fusion protein to the plasma membrane (PM); and
The asterisk (*) indicates the point of random fusions.
   Expression of GFP:SPS is driven by the promoter from the trapped gene.

FIGS. 8A-8G represent potential outcomes when “trapping” genes in a diploid or haploid cell line. FIG. 8A illustrates plasma membrane (PM) localization of an S-palmitoylation substrate (SPS) fused to GFP (GFP:SPS). FIG. 8B illustrates how functionally disrupting a single allele of a critical gene in a diploid cell line could have no apparent visual affect on the PM localization of an GFP:SPS. FIG. 8C illustrates how functionally disrupting a single allele of a critical gene in a diploid cell line could have a partial affect on the PM localization of an GFP:SPS, displacing a variable amount of GFP:SPS from the PM. FIG. 8D illustrates a convenient result wherein complete displacement of GFP:SPS from the PM to the cytoplasm is achieved by mutagenizing a single allele in a diploid cell.

FIGS. 8E-8G illustrate that the likelihood of displacing a significant fraction of GFP:SPS from the PM may be increased by using a haploid (frog, Rana) cell line. Thus, FIG. 8E illustrates PM localization of GFP:SPS in wild-type haploid cells. Mutagenizing (functionally disrupting) a single allele of a critical gene in such a line would increase the likelihood of inducing a completely cytosolic localization (see FIG. 8F), except in the case where functional redundancy among members of a gene family can compensate for partial loss of function (see FIG. 8G). The functional redundancy problem would be true in the diploid cell line as well.

FIGS. 9A-9C illustrate CHO-K1 hypodiploid cells stably expressing GAP43:GFP. FIGS. A1, B1 and C1 are images of small colonies of stable cells. FIGS. A2, B2, C2 are the same images analyzed by the membrane segmentation algorithm described in grant proposal (1 R21 MH071400-01A1). The green line demarcates the plasma membrane, demonstrating that the algorithm has no problem finding the PM in CHO-K1 cells.

FIGS. 9D-9G illustrate ICR-2A haploid Frog cells stably expressing GAP43:GFP. FIGS. D1, E1, F1 and G1 are images of cells from very disperse colonies of stable cells. Figures D2, E2, F2 and G2 are the same images analyzed by the membrane segmentation algorithm described in grant proposal (1 R21 MH071400-01A1). The green line demarcates the plasma membrane, demonstrating the algorithm has no problem finding the PM in CHO-K1 cells. These clones were isolated only 2 days prior to making these images, grow slowly and as such are still very dispersed in the dish. The morphology of these cells changes for the better as they become more densely packed on the plate. These images make it clear that the HTM, membrane segmentation algorithm is competent to identify the PM of a cell, regardless of cell size, shape and density.

### DETAILED DESCRIPTION OF THE INVENTION

In accordance with the present invention, there are provided assays which result from combining gene trapping in haploid (or hypodiploid) cells with HTM. As will be appreciated by those of skill in the art, numerous biological phenomena can be beneficially explored using invention methodology. Such phenomena include prenylation (e.g., famesylation and geranylgeranylation), acylation (e.g., palmitoylation and myristoylation), regulation of transcription, the action of steroids and steroid-like compounds, neuroregeneration and spinal cord repair, cell cycle regulation, stem cells and neuronal stem cells (i.e., to enable an understanding of signal transduction pathways that determine/regulate cell developmental fate and provide a specific means by which to regulate or manipulate cell fate, cell migration, filopodia, GPCR-related signaling, phosphorylation, contact-inhibition of cell growth, tumorigenesis, identification of the molecular targets of drugs with unknown mechanism(s) of action, and the like, as well as any signaling pathway or cellular process in which a unique morphological metric can be tied (either directly or indirectly) to that process. As readily recognized by those of skill in the art, there is with near absolute certainty, some such marker for every pathway or process in a cell. By way of illustration, exemplary metrics which may be monitored to investigate the biological phenomena set forth above are presented in the following table.

TABLE

| Phenomenon of interest | Metric |
|---|---|
| Prenylation | plasma membrane (PM)<>cytoplasm translocations |
| Acylation | PM<>cytoplasm translocations |
| Regulation of transcription | Cytoplasm<>nucleus translocation |
| Action of steroids and | Cytoplasm<>nucleus translocation and/or granule |
| steroid-like compounds | formation |
| Neuroregeneration/spinal | Neurite extension and/or protein/molecular markers |
| cord repair | |
| Cell cycle regulation | DNA content, chromosomal morphology, nuclear |
| | morphology |
| Stem Cells/Neuronal stem | Specific molecular markers |
| cells. | |
| Cell migration | Tracks cleared in a fluorescent-bead layer on bottom of |
| | culture dish |
| Filopodia | Size, shape, number and distribution of labeled filopodia |
| | on cells |
| GPCR-related signaling | B-arrestin translocation/vessicular granulation |
| Phosphorylation | Phospho-specific antibody labeling/subcellular |
| | distribution or intensity of signal |
| Contact-inhibition of cell | overlap/apposition based on a suitable marker (e.g., a PM |
| growth | marker) |
| Tumorigenesis | Protein/molecular markers and or tumor formation based |
| | on cell mass, contact inhibition, overlap/aposition |
| Identification of molecular | Any metric already known to be associated with the |
| targets of drugs with | activity of the drug |
| unknown mechanism of | |
| action | |
| Any signaling pathway or | Any signaling pathway or cellular process in which a |
| cellular process in which a | unique morphological metric can be tied either directly or |
| unique morphological | indirectly to that process. There is with near absolute |
| metric can be tied either | certainty some such marker for every pathway or process |
| directly or indirectly to that | in a cell. |
| process. There is with near | |
| absolute certainty some | |
| such marker for every | |
| pathway or process in a cell. | |

All of the above-described metrics can be monitored in a variety of ways, e.g., by selective labeling of the moving/translocating, elongating/granulating, component with fluorescent proteins or antibodies or affinity tag labels (such as FlAsH, ReAsH or Hoechst; see, for example, Adams et al., 2002; Leubke, 1998; Marek and Davis, 2002; Tour et al., 2003; Griffin et al., 2000; and Park et al., 2004). The homogeneity of a given response can be dramatically increased (i.e. reduction in the biological noise component of the assay).

In accordance with a specific embodiment of the present invention, there are provided methods for identifying a gene that modulates subcellular localization of a protein, said methods comprising:
a) contacting a haploid or hypodiploid cell which expresses said protein with an insertional mutagen under conditions suitable to produce modified haploid or hypodiploid cell(s),
b) detecting a change in the subcellular localization of the protein as a result of the insertional mutagen; and
c) identifying, in those modified haploid or hypodiploid cells in which a change in the subcellular localization of the protein is detected, the gene into which the insertional mutagen is inserted, thereby identifying the gene that modulates subcellular localization of the protein.

As used herein, the term “contacting” refers to any suitable means of bringing a DNA- or RNA-based mutagenic element into physical contact with the cell and passing the mutagenic element from the exterior of the cell to the interior. As readily recognized by those of skill in the art, this can be accomplished in a variety of ways, such as, for example, by use of a viral infective particle, electroporation of naked DNA or RNA, transfection of naked DNA or RNA employing a lipid-based transfection reagent, transfection of naked DNA or RNA employing high-intensity sound or a high-density microscopic particle (e.g., gold) introduced at high-velocity (biolistic transfection), and the like.

As used herein, the term “detecting” refers to any of a variety of means that can be used to identify (e.g., quantitatively or semi-quantitatively) the occurrence of any change (or plurality of changes) of any type, no matter how subtle, that can be captured by a recording device such as a camera, fluorimeter, luminometer, photodiode, PMT, methods/photophysical phenomena (such as fluorescence resonance energy transfer, fluorescence polarization, anisotropy, fluorescence lifetimes, coloration or fluorescence resulting from an enzymatic reaction with a chromogenic or fluorigenic substrate), and the like.

As used herein, the term “identifying” refers to the delineation of any of a variety of parameters (or sets of parameters) that describe the normal or basal state of a cell population, relative to cell(s) that fall outside of the normal range. Such cells are typically subjected to further analysis. For example, cells that fall outside of the normal range may do so because of a change in any one or more morphometric properties (as described in greater detail herein). Subcategorization of the cells that have entered the non-normal population will determine whether the mutation has introduced a desired affect or not.

Stated more generally, the present invention provides methods for identifying a gene that modulates cell morphology, said methods comprising:
a) contacting a haploid or hypodiploid cell with an insertional mutagen under conditions suitable to produce modified haploid or hypodiploid cell(s),
b) detecting a change in the morphology of the cell as a result of the insertional mutagen; and
c) identifying, in those modified haploid or hypodiploid cells in which a change in cell morphology is detected, the gene into which the insertional mutagen is inserted, thereby identifying the gene that modulates cell morphology.

As used herein, “cell morphology” refers to any feature (or combination of features) of a cell (or cell population) which can be detected (by any available means), such as, for example, cell size, cell shape, cell volume, quantity and/or distribution of subcellular components (e.g., organelles, macromolecular (hetero and homo) complexes, single molecules, and the like), morphologies in the cell population as a whole (e.g., islands, spheres, spheroids, clumps, striations, waves, and the like), behaviors of the cell population as a whole (e.g., adherence, or lack of adherence to the substrate, migration of single or groups of cells, repulsive or attractive properties between a single cell or groups of cells, and the like), the stage of the cell cycle (e.g., G0, G1, G2, S or M phases), the degree of cell differentiation, and the like.

In accordance with another specific embodiment of the present invention, there are provided methods for identifying a gene that modulates subcellular localization of a protein, said methods comprising:
a) contacting a haploid or hypodiploid cell which expresses said protein with an insertional mutagen under conditions suitable to produce modified haploid or hypodiploid cell(s),
b) selecting those modified haploid or hypodiploid cells which reveal a change in the subcellular localization of the protein; and
c) identifying, in the modified haploid or hypodiploid cells selected in step (b), the gene into which the insertional mutagen is inserted, thereby identifying the gene that modulates subcellular localization of the protein.

Stated more generally, the above-described embodiment of present invention provides methods for identifying a gene that modulates cell morphology, said methods comprising:
a) contacting a haploid or hypodiploid cell with an insertional mutagen under conditions suitable to produce modified haploid or hypodiploid cell(s),
b) selecting those modified haploid or hypodiploid cells which reveal a change in cell morphology; and
c) identifying, in the modified haploid or hypodiploid cells selected in step (b), the gene into which the insertional mutagen is inserted, thereby identifying the gene that modulates cell morphology.

In accordance with an alternative embodiment of the present invention, there are provided methods for identifying a gene that modulates subcellular localization of a protein, said methods comprising:
a) selecting modified haploid or hypodiploid cell(s) which express said protein and which reveal a change in the subcellular localization of the protein upon contacting with an insertional mutagen, and
b) identifying, in the modified haploid or hypodiploid cells selected in step (a), the gene into which the insertional mutagen is inserted, thereby identifying the gene that modulates subcellular localization of the protein.

Stated more generally, the above-described embodiment of the present invention provides methods for identifying a gene that modulates cell morphology, said methods comprising:
a) selecting modified haploid or hypodiploid cell(s) which reveal a change in cell morphology upon contacting with an insertional mutagen, and
b) identifying, in the modified haploid or hypodiploid cells selected in step (a), the gene into which the insertional mutagen is inserted, thereby identifying the gene that modulates cell morphology.

In accordance with yet another alternative embodiment of the present invention, there are provided methods for identifying a gene that modulates subcellular localization of a protein, said methods comprising:
a) contacting a haploid or hypodiploid cell which expresses said protein with an insertional mutagen under conditions suitable to produce modified haploid or hypodiploid cell(s); and
b) identifying, in those modified haploid or hypodiploid cells in which a change in the subcellular localization of the protein occurs, the gene into which the insertional mutagen is inserted, thereby identifying the gene that modulates subcellular localization of the protein.

Stated more generally, the above-described embodiment of the present invention provides methods for identifying a gene that modulates cell morphology, said methods comprising:
a) contacting a haploid or hypodiploid cell with an insertional mutagen under conditions suitable to produce modified haploid or hypodiploid cell(s); and
b) identifying, in those modified haploid or hypodiploid cells in which a change in cell morphology occurs, the gene into which the insertional mutagen is inserted, thereby identifying the gene that modulates cell morphology.

In accordance with still another alternative embodiment of the present invention, there are provided methods for identifying a gene that modulates subcellular localization of a protein, said methods comprising identifying, in those modified haploid or hypodiploid cells which reveal a change in the subcellular localization of the protein upon introduction of an insertional mutagen thereto, the gene into which the insertional mutagen is inserted, thereby identifying the gene that modulates subcellular localization of the protein.

In accordance with another aspect of the present invention, there are provided methods for identifying a gene that modulates cell morphology, said methods comprising identifying, in those modified haploid or hypodiploid cells which reveal a change in the cell morphology upon introduction of an insertional mutagen thereto, the gene into which the insertional mutagen is inserted, thereby identifying the gene that modulates cell morphology.

In accordance with yet another aspect of the present invention, there are provided methods for determining the enzymatic cascade of genes responsible for a protein modification of interest, said methods comprising:
randomly mutagenizing a haploid or hypodiploid cell, optionally containing one or more stably expressed marker gene(s), with a mutagenic element,
selecting/detecting those cell lines that harbor the mutagenic element, and optionally one or more stably expressed marker gene(s), and
identifying, in the modified haploid or hypodiploid cells selected/detected above, a change in phenotype, thereby identifying a gene in the enzymatic cascade of interest.

In accordance with an alternate embodiment of the present invention, there are provided methods for determining the enzymatic cascade of genes responsible for a protein modification of interest, said methods comprising:
selecting/detecting those haploid or hypodiploid cells, optionally containing one or more stably expressed marker gene(s), that harbor a mutagenic element, and optionally one or more stably expressed marker gene(s) as a result of being randomly mutagenized with a mutagenic element, and
identifying, in the modified haploid or hypodiploid cells selected/detected above, a change in phenotype, thereby identifying a gene in the enzymatic cascade of interest.

In accordance with another alternative embodiment of the present invention, there are provided methods for determining the enzymatic cascade of genes responsible for a protein modification of interest, said methods comprising identifying a change in phenotype in modified haploid or hypodiploid cells prepared by randomly mutagenizing a haploid or hypodiploid cell, optionally containing one or more stably expressed marker gene(s), with a mutagenic element, thereby identifying a gene in the enzymatic cascade of interest.

In accordance with still another aspect of the present invention, there are provided stable, haploid or hypodiploid lines expressing a detectable marker, operably associated with a substrate for a reaction of interest. As readily recognized by those of skill in the art, a variety of detectable markers can be employed herein, such as, for example, a fluorophore, a chromophore, a chromogenic substrate, and the like.

Presently preferred detectable markers contemplated for use herein are fluorophores, such as Green Fluorescent Protein (GFP).

As readily recognized by those of skill in the art, invention cell lines can be employed to study a wide variety of reactions of interest, such as, for example, palmitoylation, prenylation, acylation, regulation of transcription, the action of steroids and steroid-like compounds, neuroregeneration and spinal cord repair, cell cycle regulation, stem cells and neuronal stem cells, cell migration, filopodia, GPCR-related signaling, phosphorylation, contact-inhibition of cell growth, tumorigenesis, identification of the molecular targets of drugs with unknown mechanism(s) of action, and any signaling pathway or cellular process in which a unique morphological metric can be tied (either directly or indirectly) to that process, and the like.

For example, when the reaction of interest is palmitoylation, the substrate employed herein will typically be a short peptide, such as, for example, an S-palmitoylation substrate.

Recent advances in machine visions have resulted in an explosion of the application of imaging to cell biology. Several companies including Q3DM (San Diego, Calif.) have developed microscopic platforms and machine vision algorithms having such a degree of sophistication that they are being implemented by major pharmaceutical companies for mainstream drug discovery and by academic labs with a need for extremely quantitative analyses of morphological events in cell biology. The combination of functional genomics like gene trapping and HCM has tremendous untapped potential.

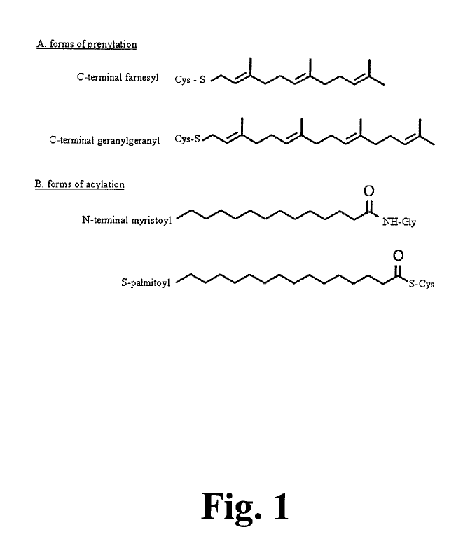

An example shows how useful the combination of a haploid cell line and HCM is (see also FIGS. 1A and 1B). The enzymatic cascade of genes responsible for the various forms of protein palmitoylation (a post-translational modification of proteins that causes the host protein to be associated with a cellular membrane, commonly the plasma membrane) can be determined. Briefly, this is accomplished by creating a stable, haploid line expressing Green Fluorescent Protein (GFP) genetically fused to a short peptide substrate (S-palmitoylation substrate: SPS—in this case) for palmitoylation or any lipidation reaction of interest. This stable cell line is then randomly mutagenized, or “gene trapped” using a standard mutagenic element and procedures. Selective pressure (e.g., antibiotic resistance) can be employed to enrich the cell pool for tens to hundreds of thousands of individual cell lines that harbor both the mutagenic element and the stably expressed GFP:SPS transgene. If the mutagenic element disrupts a gene directly (or indirectly) responsible for the addition of the lipid adduct to the GFP:SPS substrate, the fluorescent reporter of lipidation, GFP:SPS, will no longer be localized to the cellular membrane as it would under normal circumstances. Rather the protein will be localized to the cytoplasm as non-palmitoylated GFP is normally.

Determining the genes into which the mutagenic element was introduced, resulting in the redistribution of GFP from the membrane to the cytoplasm, is readily accomplished, employing standard procedures. The task of screening tens of thousands of individual mutated cell lines for a gross change in the distribution of a fluorescent reporter like GFP is readily accomplished employing an HCM system like the EIDAQ100 from Q3DM.

Existing algorithms allow for discriminating morphological details such as the redistribution of a fluorescent reporter from the membrane to the cytoplasm. Such algorithms are also capable of discriminating interesting mutants from mutants benign to the morphology of interest with enough speed to completely assay the entire genome three times per day (assuming that the genome consists of roughly 30,000 unique genes). In contrast, this process would take many months of expensive labor if scoring such changes by eye.

The process described above can readily be applied to any case where there is a change in physiology or morphology and an algorithm exists that can describe and quantitate the event. A large library of metrics currently exists, making it relatively simple to tailor a system to ones needs. The combination of HCM with the use of haploid or hypodiploid cells is completely unique and has the potential to rapidly elucidate all steps of signaling cascades that regulate processes that can be detected using fluorescence microscopy.

Many proteins are concentrated on the plasma membrane (PM), trapped in specialized subcellular regions, like synapses and caveolae, by virtue of their lipid modifications. Thio-acylation or S-palmitoylation, a common form of lipid modification, is unique in that it is reversible and dynamic, suggesting a modulatory role in signal transduction similar to phosphorylation. Recent data indicate that proper, dynamic regulation of the palmitoylation of PSD-95, an abundant scaffolding protein in the synapse, is critical for synaptic organization and function, linking palmitoylation to complex processes such as learning, memory and disease. Consistent with this understanding, it is known that mutations in one gene regulating S-palmitoylation result in a severe neurodegenerative disorder, infantile neuronal ceroid lipofuscinosis or ICNL. Additionally, a candidate gene for the regulation of S-palmitoylation is linked to schizophrenia. These examples highlight the importance of properly regulated S-palmitoylation to human health and disease.

Biochemical characterization of the enzymes responsible for S-palmitoylation (palmitoyl thio-acyl transferases, S-PATs) has been difficult and controversial; recent data from experiments in yeast add substantial weight to the view that such enzymes exist. To date, functional genomics discovery programs in vertebrate systems similar to those in yeast have been expensive and time consuming. The present invention addresses this problem by combining a novel form of gene-trapping in vertebrate cell cultures, with a fully automated readout in a high-throughput microscopy (HTM) format. The invention assay system enables one to test directly and functionally for the presence of S-PATs in vertebrates. The present invention further enables one to elucidate the entire enzymatic pathway for protein S-palmitoylation by quantitatively analyzing millions of cells from tens of thousands of “trapped” cell lines.

The system described herein, using S-palmitoylation as an exemplary pathway, provides critical information about the regulation of S-palmitoylation. The invention system also provides an invaluable experimental tool that can be extended to screens for other genes that regulate the subcellular distribution and concentration of proteins, enabling numerous applications in basic and therapeutic research.

Many proteins are concentrated on the PM by virtue of their lipid modifications. Recent data show that lipid modifications of proteins may well be the primary physical determinant for targeting to and retention of some proteins to membrane lipid microdomains such as synapses and caveolae (El-Husseini et al., 2000; El-Husseini Ael et al., 2002; Kanaani et al., 2002; Loranger & Linder, 2002; Topinka & Bredt, 1998; Zacharias et al., 2002). Fusion of Green Fluorescent Protein (GFP) to small-peptide substrates for lipid modification (e.g. the N-terminal 18 residues of GAP-43) has been shown to be sufficient to localize the fusion proteins to the PM in the absence of any other targeting signal (Zacharias et al., 2002). Similarly, mutagenic substitution of modifiable residues for ones which cannot be modified results in gross mislocalization and/or loss of function of the expressed proteins (Craven et al., 1999; Hiol et al., 2003; Osterhout et al., 2003; Wiedmer et al., 2003). It has also been shown that different lipid moieties induce partitioning into different lipid environments or lipid microdomains of cells (Melkonian et al., 1999; Moffett et al., 2000; Zacharias et al., 2002). Specific associations of proteins within such microdomains, whether mediated by attractive, protein-protein interactions, forced proximity or both, are critical components in the architecture of cellular communication. Lipid modifications undoubtedly play an important role in the creation and modulation of such protein associations. This point is amply demonstrated by the fact that the regulation of protein lipidation is known to be involved in several forms of cancer (Dinsmore & Bell, 2003; Ghobrial & Adjei, 2002), a severe neurodegenerative disorder, ICNL (Vesa et al., 1995) and possibly for schizophrenia (Liu et al., 2002). New optical methods and fluorescent sensors for characterizing the state of protein lipidation, protein localization and protein interactions (like those described herein) are consistently at the technological forefront, driving new discoveries in these fields and providing insight into the intricate, structured workings within cells and in networks of cells (Lippincott-Schwartz & Patterson, 2003; Weijer, 2003; Zacharias et al., 2000).

Prenyl and Acyl groups are the most common forms of protein lipid modifications (see FIGS. 1A and 1B). The two most common forms of prenylation are geranylgeranylation and famesylation (FIG. 1A) while myristoylation and palmitoylation (FIG. 1B) are likely the most common forms of acylation. Most, if not all of the biochemical steps regulating the prenylation of proteins have been deciphered (reviewed in (Fu & Casey, 1999; Roskoski, 2003; Sinensky, 2000)). In fact, the mechanistic pathway for famesylation has been determined to the atomic level (Long et al., 2002). This density of information for prenylation is due in part to the fact that prenyltransferases have been fairly successful therapeutic targets for the treatment of several types of cancer (Dinsmore & Bell, 2003; Ghobrial & Adjei, 2002), underscoring the importance of protein lipidation for human health and disease.

In contrast to prenylation, a detailed knowledge about the process of protein acylation is still lacking. Among the types of acylation, enzymatic processes regulating myristoylation have been characterized best and are reviewed in (Farazi et al., 2001; Rajala et al., 2000; Resh, 1999). Briefly, proteins that will become myristoylated begin with a consensus sequence Met-Gly-X-X-X-Ser/Thr (SEQ ID NO:4). The start Met is co-translationally, proteolytically removed and the myristate is added to the exposed N-terminal glycine via a stable amide bond. The formation of this bond is catalyzed by N-myristoyl transferase with a high degree of selectivity for 14-carbon myristate (Farazi et al., 2001; Rajala et al., 2000). N-terminal myristoylation often exists in combination with palmitoylation which can take at least two forms: N-palmitoylation (apparently rare) and S-palmitoylation (the most common). N-palmitoylation, first described for the protein sonic hedgehog (Pepinsky et al., 1998), is the addition of palmitic acid to the a-amide of Cys-24, which is proteolytically exposed to become the N-terminal residue of the functional protein. Addition of a palmitoyl group by an amide bond to the N-terminal glycine was recently shown to occur on the heterotrimeric G-protein, Gas (Kleuss & Krause, 2003). In contrast, S-palmitoylation is a reversible modification that occurs via a labile thioester bond with the thiol side chains of reactive cysteine residues on the cytoplasmic portions of a protein. Proteins that are palmitoylated are relatively abundant and diverse (Melkonian et al., 1999; Moffett et al., 2000) and the functional consequences of the modification vary depending on the protein. In general, palmitoylation increases the hydrophobicity of a protein, thereby affecting the degree of membrane association as well as sublocalization within a membrane. Once associated with the membrane, the palmitoyl group partitions primarily into cholesterol- and sphingolipid-rich lipid rafts (Moffett et al., 2000; Zacharias et al., 2002). The additional membrane avidity increases the likelihood that the palmitoylated protein will interact (forced proximity) with other membrane-bound or membrane-associated proteins, a phenomenon that is exemplified by the synaptic scaffolding protein, PSD-95 (Craven et al., 1999; El-Husseini et al., 2000; Perez & Bredt, 1998; Topinka & Bredt, 1998). The resulting complexes of interacting proteins assembled at sites of membrane specialization like synapses are critical nodes containing signaling proteins that are involved in every conceivable signaling pathway. Furthermore, modulation of the associative properties of individual proteins in these networks by reversibly palmitoylating some members, is a very attractive and plausible mechanism to regulate the participation of proteins in signaling events (el-Husseini Ael & Bredt, 2002; El-Husseini Ael et al., 2002; Hess et al., 1993; Qanbar & Bouvier, 2003).

The finding that the residence half life of the palmitoyl group on proteins is considerably shorter than the half life of the proteins to which it is attached (Lane & Liu, 1997; Wolven et al., 1997) suggests that enzymes for palmitate removal, protein palmitoylthioesterases or PPTases, could exist. PPT1 (Camp & Hofmann, 1993; Camp et al., 1994) is a lysosomal hydrolase that participates in the degradation of palmitoylated proteins by deacylating cysteine thioesters; acyl protein thioesterase 1 (APT1), a cytoplasmic protein first biochemically characterized as an acyl thioesterase by Duncan and Gilman (1998), is a member of the serine hydrolase, α/β fold family of lysophospholipases that has several additional substrate and lipid specificities (Duncan & Gilman, 1998). Regulated removal of the palmitoyl group from proteins is critical in human health; defects in PPT1 result in a severe neurodegenerative disorder known as infantile neuronal ceroid lipofuscinosis (ICNL) (Vesa et al., 1995). The role of PPT1 in ICNL was confirmed by targeted disruption of the gene in a mouse resulting in a model of the human disorder (Gupta et al., 2001).

In spite of the fact that the mechanism for depalmitoylation is reasonably well understood, elucidation of the mechanism for regulation of protein S-palmitoylation has lagged significantly behind. In one aspect of the present invention, elucidation of the enzymatic pathway that leads to protein palmitoylation is provided. There is currently no known amino acid consensus sequence for palmitoylation, suggesting either:
1) a non enzymatic mechanism for palmitoylation, or equally as likely
2) the existence of multiple, unidentified enzymes whose homo/hetero stoichiometry, cofactors or other undefined factors are unknown.
   Palmitoyl-CoA can spontaneously acylate cysteine residues of some fragments of full length proteins that are normally palmitoylated (Bharadwaj & Bizzozero, 1995; Quesnel & Silvius, 1994) and some, but not all, fully-folded, normally-acylated proteins. (Duncan & Gilman, 1996). For example, myristoylated G_{iα1} is efficiently and stoichiometrically auto-palmitoylated on Cys³ (Duncan & Gilman, 1996). However, in the same study, SNAP-25, GAP-43 and Fyn kinase were not spontaneously acylated. Since the mid 1980s, biochemists have been able to purify, to varying degrees, S-PAT activity from different cell and membrane types (Berger & Schmidt, 1984; Berthiaume & Resh, 1995; Dunphy et al., 1996; Mack et al., 1987). The biochemical characterization of these proteins has been difficult and has, in some cases, led to the discovery of proteins that were involved in lipid metabolism rather than the transfer of palmitoyl groups. Using genetic approaches in yeast, two proteins with S-PAT activity have been identified and characterized: Erf2p (Bartels et al., 1999; Dong et al., 2003; Long et al., 2002) and Akrlp (Roth et al., 2002) (reviewed by Linder and Deschenes (2003)). The Erf2p coding sequence predicts four transmembrane spanning domains and Arkp1, six transmembrane spanning domains. Both proteins contain DHHC-CRD domains (i.e., Asp-His-His-Cys-Cysteine Rich Domain; SEQ ID NO:5) (Putilina et al., 1999) thought to be responsible for the S-PAT activity as mutations in the DHHC domain abolish S-PAT activity (Bartels et al., 1999) and because homology between these two proteins, both with S-PAT activity, is limited to the DHHC-CRD domains. Orthologous proteins exist in every eukaryote examined so far (Linder & Deschenes, 2003), emphasizing the fundamental importance of S-palmitoylation. It is also important to note that a protein containing a DHHC-CRD motif and highly homologous to Erf2p was identified as a candidate gene for schizophrenia (Liu et al., 2002). Whether the candidate gene encodes an S-PAT remains to be seen, but if so it raises the possibility that pharmacologically modulating S-PAT activity in the brain could result in a treatment for schizophrenia. Development of the methods described herein form the basis for a high-throughput screen for small-molecule modulators of S-PAT activity.

### Functional Genomics and High Throughput Microscopy as Methods for Studying Signal Transduction

Human orthologs of yeast proteins have been identified from database queries. It remains to be seen, however, whether all DHHC-CRD proteins exhibit S-PAT activity, and, if so, whether they act alone, as hetero/homo-oligomers, or in concert with cofactors or other proteins. The first successful identification of these proteins took advantage of the genetic tractability of yeast. Similar genetic manipulations in vertebrate systems such as mice are far more labor intensive but often times required, for example when the mutant phenotype affects the development of the organism. However, when a mutation is expected to cause a visually identifiable change in the concentration or distribution of a protein, it becomes possible to reduce the experimental model to one where the gene of interest is mutated in a cultured cell line and the phenotype analyzed using microscopy. Specifically, using gene- and/or promoter-trap mutagenesis and examining the resulting mutant cell lines using HTM, would enable cell-based experimental analysis of gene function, on a genome-wide scale. This nascent technology can be employed to rapidly (on the order of hours) screen tens of thousands of mutated, stable cell lines covering millions of cells, for one or more mutants having a phenotype that is indicative of a gene that is directly, or indirectly, involved in regulating protein distribution or concentration. Proteins contemplated for use in the practice of the present invention would be those involved in the addition of palmitates to a protein(s) or peptide responsible for the biosynthesis of palmitate, palmitate-CoA. The morphological metric of interest in this case will be the subcellular localization of the reporter, Green Fluorescent Protein (GFP) fused to an S-palmitoylation substrate (SPS); GFP:SPS. Briefly, under normal circumstances GFP:SPS would be localized at the PM. However, if the reporter construct mutates a gene in the signaling pathway for S-palmitoylation, GFP:SPS will relocalize from the PM to the cytoplasm.

### Gene Trapping as a Method to Identify S-PATs

Gene- and promoter-trapping are forms of insertional mutagenesis whereby reporter genes and/or selectable markers are randomly and likely stochastically (Chowdhury et al., 1997; Evans, 1998) inserted into the genome of mouse ES cells (reviewed in: Cecconi & Meyer, 2000; Cecconi & Gruss, 2002; Stanford et al., 2001). Traditionally functional genomics studies employing gene-trap have relied on “trapping” the genes in ES cells, generating lines of mice with the mutated ES cell lines, then analyzing the phenotype resulting from the mutation in the whole animal (Evans et al., 1997; Stanford et al., 2001). This approach, while very informative and necessary in many cases (such as the role of the gene in the development of the animal), is labor intensive and expensive. While the methods of the present invention do not seek to circumvent or replace the process of screening mutant phenotypes in mice when it is necessary, instead the invention methods provide:
1) a screening format that enables the dissection of signaling pathways at the cellular level using gene-trap technology, and
2) an enabling technological platform wherein the prescreening of ES cells for a desired mutation enhances the process of generating mutant mice by pre-selecting mutant ES cells displaying a desired or predictive, characteristic phenotype.

In accordance with the present invention, HTM (also referred to herein as high-content screening or HCS) is adapted to the functional genomics format, thereby enabling the screening of thousands of cell lines to determine the subcellular localization of the GFP:SPS reporter. HTM is a technology just now coming of age in drug discovery research (Milligan, 2003; Price et al., 2002; Woollacott & Simpson, 2001) in pharmaceutical companies. There is a vast and untapped potential for this technology stemming primarily from the fact that this platform provides the most quantitative mechanism for doing cell biological experiments of virtually any type (Price et al., 2002). Traditionally, the method for determining subcellular localization of proteins has been visual inspection and interpretation of relatively low numbers of microscopic images of cells expressing a GFP-tagged protein or fluorescent-labeled antibodies with specificity to a protein (Giuliano & Taylor, 1998; Zacharias et al., 2000). However, such subjective interpretations may be unconsciously or consciously influenced by investigator bias. Data obtained this way are not always easily confirmed in independent investigations, are not usually amenable to statistical analysis and are labor intensive. A primary motivating factor in the development of HTM has been the perceived need of pharmaceutical companies (Taylor et al., 2001) to investigate rapidly the effects of millions of small molecule compounds on a target molecule within the complex environment of a living cell; an environment where entire, endogenous intracellular signaling pathways are hopefully intact and functional. It has been the hope of the developers of HTM and the pharmaceutical industry that the increased information density that can be generated using multiplexed fluorescent readouts of drug responses would increase the quality of “hits” in a primary (>a million compounds) screen as well as decrease the time required to determine if lead compounds were functioning via the desired or predicted signaling routes. This type of screening method will prove very useful to the pharmaceutical industry, as well as in basic, academic research (Yarrow et al., 2003). HTM will add a badly needed quantitative aspect to cell biology.

Morphometric Analysis with HTM

Existing HTM algorithms are capable of discriminating minute changes in cell morphology or subcellular protein distribution (Conway et al., 2001; Ghosh et al., 2000; Minguez et al., 2002). There is essentially no limitation on the types of morphology that can be used, alone or combinatorially, as the criteria for a unique marker (Boland & Murphy, 2001; Price et al., 1996; Price et al., 2002; Roques & Murphy, 2002). Multiple criteria ranging from the location or concentration of a fluorophore in a cell (Boland et al., 1998; Boland & Murphy, 1999 a,b;2001; Markey et al., 1999; Murphy et al., 2000; Price et al., 2002; Roques & Murphy, 2002) to a physical change in the shape of a cell or a redistribution of cellular contents such as chromosomes, transcription factors (Ding et al., 1998) microtubules (Minguez et al., 2002) membrane protrusions (e.g. neurites, ruffles etc)((Price et al., 1996; Roques & Murphy, 2002)) can be used individually or combined to enhance the sensitivity and accuracy (Boland & Murphy, 2001; Price et al., 1996; Roques & Murphy, 2002). These algorithms provide a flexible and unbiased determination of the existence and degree of interesting, visible change(s) in cells allowing for high-resolution determinations of pharmacological efficacy tied to such a change (Conway et al., 2001; Ding et al., 1998; Ghosh et al., 2000). It is the exquisite sensitivity for subtle change and the objectivity of HTM that make possible the use of cell lines for a wider variety of gene trap screens.

In accordance with the present invention, there are provided cell-based, high-throughput, functional genomics assays that identify genes/enzymes comprising the pathway leading to S-palmitoylation of multiple S-palmitoylation substrates. Based on the above-described assay, one of skill in the art would readily recognize that the use of HTM in cell-based, functional genomics can be expanded in that it will be the basis upon which the development of other changes in cell morphology or fluorescent probe (re)distribution can be applied to gene-trap and functional genomics.

In accordance with the present invention, there is also provided a conceptual and technical basis for developing a drug discovery screen for small-molecule modulators of protein S-palmitoylation.

Insertional mutagenesis, or gene trap, is normally performed in embryonic stem (ES) cells which are subsequently used to create mutant mouse lines in which the resulting phenotypes are analyzed. In accordance with the present invention, there is provided a novel, complementary format for screening interesting mutations in cell lines using fluorescent reporters of protein localization. Also provided are gene trap vectors that enable the discovery of genes involved in regulating protein S-palmitoylation, as well as other signaling pathways of interest. Still further provided in accordance with the present invention is methodology for the functional characterization of mutant cell lines which are characterized employing invention high-throughput methodology.

This invention is further illustrated by the following examples which should not be construed as limiting. The contents of all references, patents and published patent applications cited throughout this application, as well as the sequence listing and the figures, are incorporated herein by reference.

### EXAMPLE 1

### Preliminary Studies

To demonstrate the feasibility of finding S-PATs using a fluorescent reporter of palmitoylation, in combination with gene trapping and HTM, it is useful to establish that palmitoylation is a necessary and sufficient condition to cause the Green Fluorescent Protein: S-Palmitoylation Substrate (GFP:SPS) reporter to be localized to the plasma membrane (PM). Having this ability allows one to separate the role of palmitoylation from the influence of other signals, like protein-protein interactions, that might alter subcellular targeting. Though an example where S-palmitoylation is the exclusive physical property directing subcellular localization may not exist naturally, creating it with the reporters described herein allows one to look specifically and exclusively for modulators of S-palmitoylation of this specific, exemplary substrate.

In order to further demonstrate the feasibility of invention methods, it is also useful to demonstrate that the signaling cascade leading to palmitoylation is intact in haploid Rana ICR-2A cells (a stable cell line created from an androgenetic haploid embryo of the frog Rana pipien ATCC #CCL-145), that a pantropic retrovirus can infect ICR-2A cells and that it is possible to quantitatively analyze images of millions of cells and to determine the subcellular distribution of specific reporters, thereby raising the proposed screen to a high- throughput level that is sufficient to saturate the genome quickly and easily.

### EXAMPLE 2

### Palmitoylation is Necessary and Sufficient to Cause Localization of GAP43:GFP to the PM

There are many cases in which multiple targeting signals within a full-length protein work in concert to determine its final, subcellular location. Often, protein-protein interaction domains such as PDZ domains are found in close, linear and/or structural proximity to palmitoylatable cysteines of the same protein. A prime example of this is PSD-95 (Craven et al., 1999; El-Husseini et al., 2000; Topinka & Bredt, 1998). Isolating the portion of such a protein that is acting as the SPS is an ideal way to create a sensor specific for palmitoylation rather than for the function of the original, full-length protein from which the peptide was borrowed.

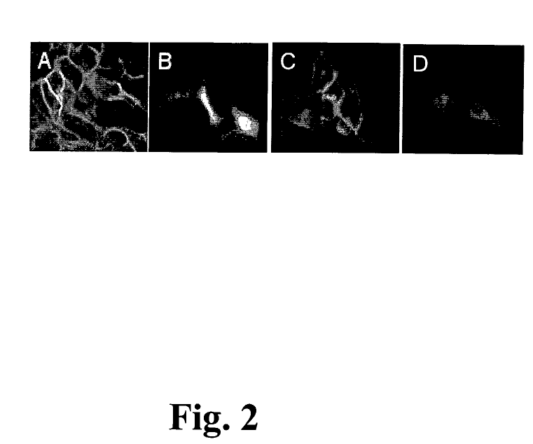

The N-terminus of GAP-43 is doubly palmitoylated on two adjacent cysteine residues. When an 18-residue S-palmitoylation substrate peptide from the N-terminus GAP43 is fused to the N-terminus of GFP, this peptide alone, by virtue of its palmitoylation, retains GFP on the PM (see FIGS. 2A-2D). Conversely if palmitoylation of the SPS peptide fused to GFP is blocked or inhibited, the protein diffuses freely throughout the cell, including the nucleus (see FIG. 2D), as is the case when GFP is not fused to any other peptide or protein (see FIG. 2B). This point is important in that if this were not so, the potential for disrupting genes not related to palmitoylation, but still causing redistribution from the PM to the cytoplasm, would be much higher. For example, one could imagine that chaperones or transport proteins that might be necessary for the normal trafficking of the original, full-length, palmitoylated protein would, when disrupted, result in redistribution of the reporter from the PM to the cytoplasm. The GAP43:GFP reporter is free of such ancillary signals for PM targeting or any enzymatic activity present in the parent protein.

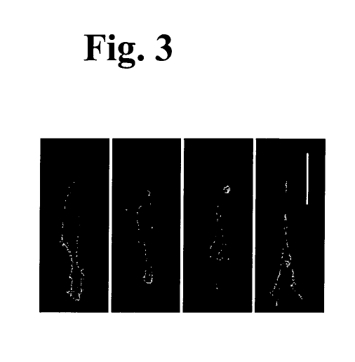

The fundamental ability to quantitatively recognize GFP localized to the PM has already been achieved by the engineers at Q3DM using methodology described herein. Computer-generated demarcation of the PM and the nucleus are demonstrated in FIG. 3. While adequate, in its current state, to meet the needs of the proposed experiments, this algorithm has not been optimized to its fullest potential.

### EXAMPLE 3

### The Enzymatic Pathway for S-palmitoylation of GAP43:GFP is Intact in ICR-2A Cells

The palmitoylation sensor, GAP43:GFP, is seen in FIG. 3 to localize appropriately to the PM of haploid ICR-2A cells, illustrating with these confocal images that a pathway leading to palmitoylation of the sensor is intact in the cell line. GAP43:GFP is expressed transiently under the control of a CMV promoter in the expression plasmid pcDNA3 (InVitrogen). Scale bar=50 μm.

### EXAMPLE 4

### Pantropic Retrovirus Infects ICR-2A Cells Directing Expression of a Positive Control, GAP43:GFP

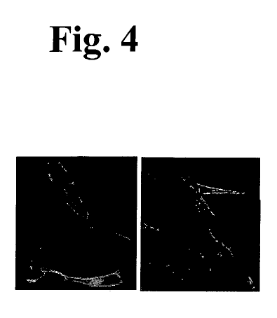

ICR-2A cells expressing GAP43:GFP, introduced by pantropic retroviral infection, are illustrated in FIG. 4. The viral titer in this experiment was lower than hoped, therefore not every cell in the field of view expressed the construct. Expression was driven by the 5′LTR/MoMuLV promoter which has given a lower expression level than what is achieved by expressing the same cDNA in these same cells under the control of the CMV promoter (see FIG. 7).

The ability to infect ICR-2A cells with retrovirus demonstrates at the most fundamental level that invention methods are applicable to mutagenesis. Likewise, expression of GAP43:GFP has been achieved by electroporating the cells with a CMV-driven expression plasmid containing the CDNA for GAP43:GFP.

### EXAMPLE 5

### The Machine Vision Algorithm Can Determine Precisely the Subcellular Localization of GAP43:GFP

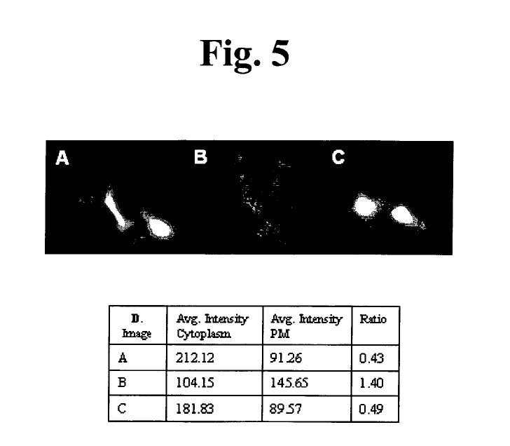

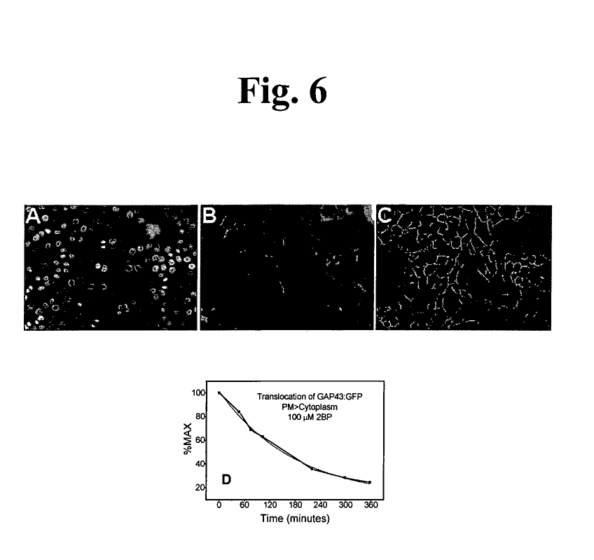

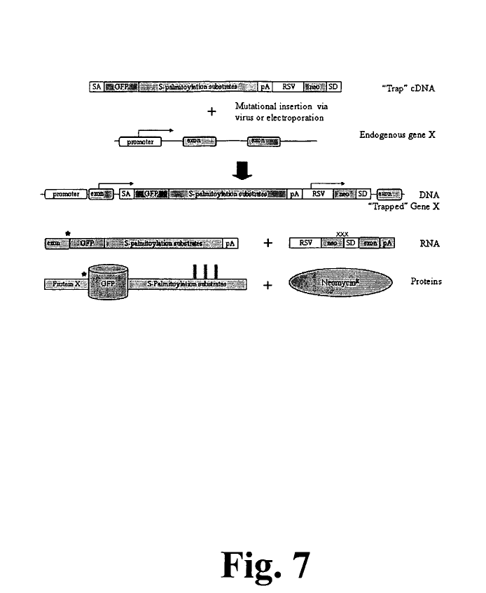

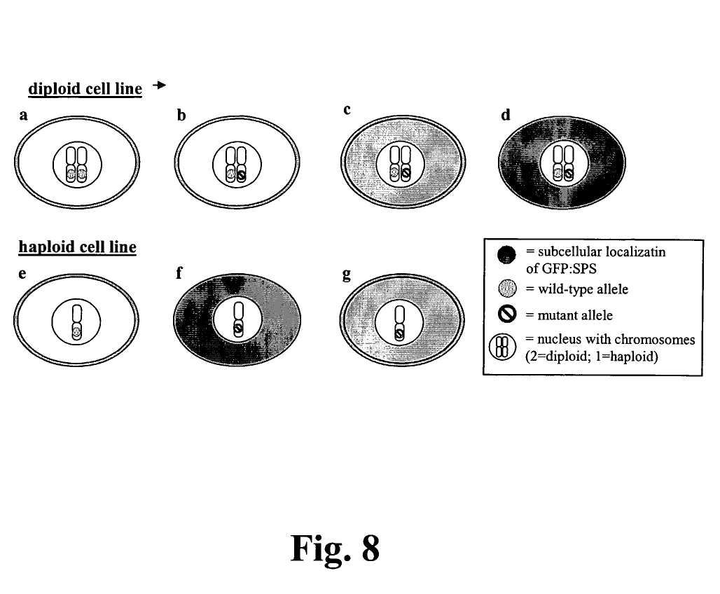

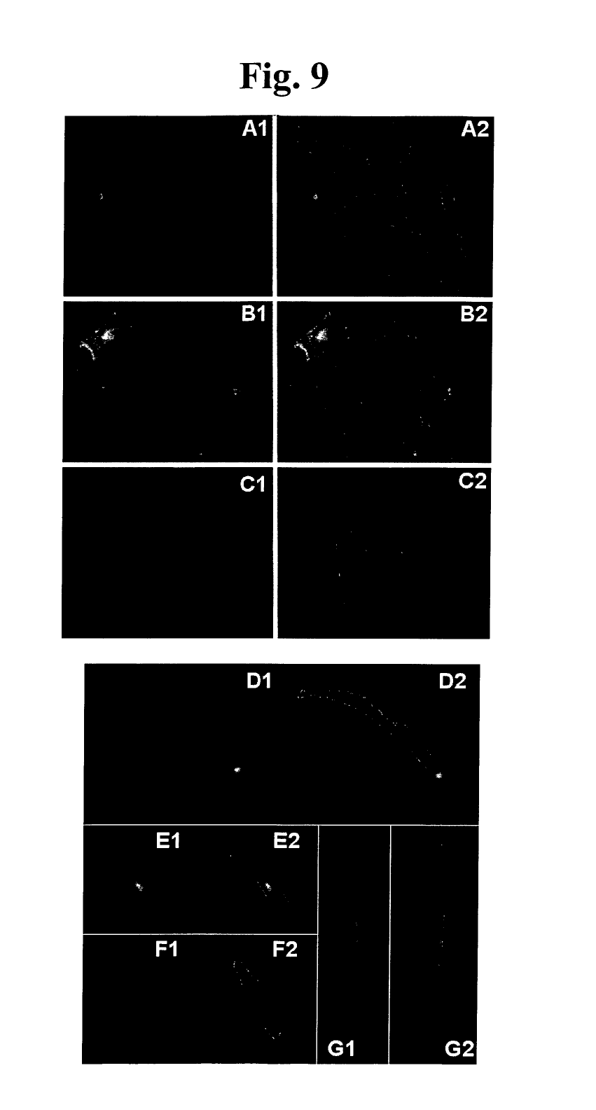

This algorithm is able to make a simple binary decision of whether GAP43:GFP is on the PM or in the cytoplasm (see FIG. 5) as well as fine incremental determinations (see FIG. 6) of the quantity of fluorescence on the PM, thereby extending the capability beyond what is necessary to score the primary screen for trapped genes. Two important questions are answered here. Data in FIGS. 5A-5D illustrate that the algorithm is sufficient to ensure success in a screen to find genes critical in the pathway for palmitoylation of the reporter in an ICR-2A reporter cell line.

The second level of sophistication, the fractional localization of the reporter to the PM, provides an unprecedented degree of precision at high throughput that makes it possible to determine, when there has been only a partial loss of function of a gene, as may be the case when one of two alleles of a gene has been mutated in a diploid cell line. Additionally, replication of previous determinations of the residence half-life of palmitate on a protein (see FIG. 6D) further validates the accuracy and broadens the utility of the algorithm. Below are representative examples of relevant analyses.

### EXAMPLE 6

### Vector Design and Introduction of DNA Into Host Cell Lines

A schematic of the vectors to be used and their resulting protein fusion products is shown in FIG. 7. All vectors can be made using standard molecular biology techniques. The vectors chosen fall into the polyA-trap (Niwa et al., 1993; Salminen et al., 1998; Voss et al., 1998; Zambrowicz et al., 1998) class in which a splice acceptor site (SA) from the engrailed-2 gene immediately precedes the promoterless reporter gene, GFP, fused to a S-palmitoylation substrate (GFP:SPS). The unique sequences provided by the SA and GFP provide primer sites for 5′ RACE. This unit is combined with the gene for neomycin resistance under the independent and constitutive control of its own, RSV promoter. A splice donor (SD) (Zambrowicz et al., 1998) at the 3′ end of the NeoR gene enables connection to the polyA tail of the trapped gene. The SD also contains stop codons in all three frames (to prevent C-terminal fusions to the NeoR protein) and unique sequence that will facilitate 3′RACE analysis of the trapped gene as well as the increase the structural integrity of the integrated reporters. An additional advantage of this configuration is that G-418 selection should be possible only when the polyA-trap vector integrates upstream of a splice acceptor and a poly-A site of an endogenous gene; intergenic insertions will be eliminated.

### EXAMPLE 7

### Methods for Integrating DNA into the Genome of the Host Cell Line

The two most common methods used to introduce the mutagenic DNA to the genome are electroporation of plasmid DNA (Chowdhury et al., 1997; Wurst & Joyner, 1993) and by virus-mediated (most commonly retrovirus) infection (Friedrich & Soriano, 1991; Zambrowicz et al., 1998). Each method has advantages and disadvantages but a general consensus is developing that a combination of these two methods is required for complete coverage of the genome (Stanford et al., 2001). The plasmid DNA used for electroporation is based in the promoterless pBluescript (Stratagene) vector and introduced into cells using the BioRad GenePulser. For retroviral infection, the Pantropic Retroviral Expression System (BD Biosciences Clontech), which efficiently infects mammalian and nonmammalian hosts including amphibians (Rana and Xenopus) is used as starting material and modified as illustrated in FIG. 3. This system uses VSV-G, an envelope glycoprotein from the vesicular stomatitis virus that is not dependent on a cell surface receptor but rather mediates viral entry through lipid binding and PM fusion (Emi et al., 1991). A modified version of the vector pLXRN (BD Biosciences Clontech) is used to express the reporter of localization GFP:SPS as well as the neomycin resistance gene. Following introduction of the mutagenic DNA by either method, clonal lines are generated by exposing the cells to G-418. G-418-resistant colonies can either be pooled and replated using limiting dilution (a method that will limit the number of clones to approximately one per well of a multiwell plate), or sorted based on their GFP fluorescence by a FACS and plated at a density of clone per well of a multiwell plate.

The trapping vectors are designed to serve the essential basic purposes required herein:
1) they provide a mechanism to determine whether a cell line has integrated a copy of the reporter (the fluorescence of GFP) and, simultaneously
2) provide a functional indicator, by virtue of its subcellular localization, for whether the reporter has mutagenized a gene necessary for directing the S-palmitoylation of the SPS.
   Integration of the gene into a locus involved in S-palmitoylation will result in fluorescence being redistributed from the PM to the cytoplasm. Using the mutagenic element as a fluorescent reporter of protein localization is a novel, and broadly applicable component of the research design. Cell lines expressing a cytoplasmic distribution are candidates for further characterization. Most cell lines express prominent labeling of the PM (as in FIG. 2A; preliminary results) with some background expression on endomembranes, as is common for this type of expression system (Zacharias et al., 2002).

The first set of experiments utilize gene-trap vectors that include a SA site fused to the 5′ end of the reporter construct (see FIG. 3). When a reporter gene is preceded by an SA, the gene must be inserted into an intron to be expressed; this method will not trap genes without introns. While this group of genes, including olfactory receptors/GPCRs (reviewed in Gentles & Karlin, 1999; Sosinsky et al., 2000), and interferons (Roberts et al., 1998), is relatively smaller than the group with introns (Gentles & Karlin, 1999), this genomic space is important and must also be surveyed. Promoter traps (e.g., Hicks et al., 1997) are appropriate tools to identify single-exon genes, and will be incorporated into the experimental program as needed following the initial screens using the polyA-trap vectors.

It is possible that the cDNA encoding the reporters could be physically fractured during the integration event (Voss et al., 1998) giving rise to the possibility that one will be integrated independently from the others. For example it could be the case that GFP becomes separated from the SPS resulting in a completely cytoplasmic pattern for fluorescence localization, or in other words, a false-positive result indicating integration into a gene necessary for S-palmitoylation. For this reason, all clonal lines reporting a positive result are further surveyed for expression of an un-fragmented reporter, GFP:SPS. Reverse transcription-PCR and/or PCR of genomic DNA using primers to the 5′ end of GFP and the 3′ end of the SPS are efficient ways to determine the integrity of the integrated reporter.

### EXAMPLE 8

### Selection and Cloning of Appropriate S-palmitoylation Substrates

S-palmitoylation substrates are cloned from whole mouse brain mRNA by RT-PCR. Fusions of the SPSs to GFP can be done by PCR and confirmed by DNA sequencing. Analysis of and selection for the appropriate expression pattern as well as the transfection/infection efficiencies of various permutations of the constructs are typically made in small scale transfection experiments prior to running a full-scale screen.

The choice of which SPSs to use, and how they are fused to GFP, is based on the tolerance of both GFP and SPSs for N- and C-terminal fusions. The fluorescence properties of GFP are unlikely to be perturbed by fusions to either terminus. In the case of the trapping vectors to be used (FIG. 3), the exact nature of fusions (“protein X” in FIG. 3) to the N-terminus of GFP cannot be predicted other than to assume that the fusions will be widely variable in their structure and function. The high degree of tolerance of GFP for fusions makes it a safer candidate to host random fusions. Conversely, certain contextual requirements exist for some peptides to be permissive substrates for S-palmitoylation. Very generally, four classes of substrate exist (Linder & Deschenes, 2003):
a. transmembrane proteins,
b. dually acylated proteins,
c. exclusively palmitoylated cytoplasmic proteins and
d. mitochondrial proteins.
   Each class has a different degree of suitability with members of class c being the best for proof-of-concept experiments. A fine example of a class c protein with apparently less rigid context or environmental requirements is Yck2p (Robinson et al., 1999; Roth et al., 2002). This protein has been shown to retain plasmalemmal localization when fused to GFP. The first mutagenesis probe I will use will be GFP:Yck2p(C-terminal tail peptide: NH2-KSSKGFFSKLGCC-COOH; SEQ ID NO:6) as it is functionally and conceptually as simple as the GAP-43 example shown in FIG. 2A.

A significant fraction GFP:Yck2p fluorescence is localized to the PM as is the case for GAP-43 (see FIG. 2A), other fluorescence should be associated mostly with endomembranes, not in the cytoplasm. Since only a fragment of Yck2p will be used as the SPS, it is expected that it will retain no activity intrinsic to the native protein that could preclude adequate expression levels.

In the event that a synthetic substrate for S-palmitoylation derived from a full-length protein does not localize GFP strongly enough to the PM, additional residues from the parent protein can be included in the fusion in an attempt to restore a preferred environment or context for full S-palmitoylation. Alternatively SPS peptides borrowed from the N- or C-termini of other proteins (e.g. GRK6 (Stoffel et al., 1994) without the PDZ-binding domain, V2R(Sadeghi et al., 1997), bovine rhodopsin (Ovchinnikov Yu et al., 1988) etc.) can be fused to GFP, evaluated for subcellular expression pattern and substituted when appropriate.

### EXAMPLE 9

### Determination of Suitable Cell Lines

S-palmitoylation is a highly conserved function, so in that respect, most every cell type and cell line could be used.

Cell lines generated by insertional mutagenesis are screened using HTM, for 1) fluorescence, indicating that the vector has integrated into the genome and is expressed and for 2) subcellular localization. If GFP:SPS is expressed solely on the PM then the disrupted gene was presumably not essential for palmitoylation of that specific SPS. Potential outcomes for such experiments are depicted in FIG. 8. It is important to remember that palmitoylation alone can be sufficient to take a GFP:SPS the plasma membrane (Zacharias et al., 2002). For this reason, it is expected that chaperones or other transport proteins/genes that might have been necessary for normal trafficking of the full-length protein from which the SPS was derived, to the plasma membrane, will not be among the “background” (i.e non-S-PATs) genes trapped.

One of the primary advantages of virus-mediated gene infection is that it integrates only a single copy per cell genome. This advantage, while making it much easier to identify the mutated gene, virtually ensures that only one of two potential alleles of a gene will be hit in an “ideal diploid” cell line. It is possible that eliminating one of two S-PAT alleles in an ideal diploid cell will be insufficient to cause total redistribution of the GFP:SPS from the PM to the cytoplasm. However using the exquisite sensitivity provided by HTM will increase the likelihood of detecting any small changes should they occur. Similarly, electroporation of reporter plasmid DNA into cells can be controlled to reduce the likelihood of multiple integrations, but this also reduces the already-slim chances of randomly hitting both alleles of a gene within the same cell line. Most cell lines have variable numbers of chromosomes, often not resembling the normal diploid state of the organism from which it was derived. Additional copies of chromosomes increases the potential copy number of particular genes of interest thereby potentially decreasing the likelihood of generating a recognizable mutant phenotype (FIG. 8). CHO-K1 (ATCC# CCL-61) cells are stably hypodiploid, meaning they have fewer than the original allotment of chromosomes and no spurious chromosomal duplications, thereby biasing the system slightly in favor of seeing a phenotypic change in response to mutagenizing a single allele of a gene. Additionally, if necessary, it is possible to bias the system further toward a gross redistribution of reporter upon mutagenesis of a critical gene by using a haploid cell line where only a single allele for each gene is represented in the genome. A stable, haploid cell line created from an androgenetic haploid embryo of the frog Rana pipiens exists and is available from ATCC (#CCCL-145, designation ICR-2A). The growth characteristics of being adherent and fibroblast-like are appropriate for culture and microscopy. If partial redistribution from the PM to the cytoplasm occurs, the trapped lines of interest can be subjected to additional rounds of insertional mutagenesis in attempts to reduce further the PM localization by trapping or mutagenizing genes that may be compensating for the originally mutated gene. This type of strategy adds dimensionality to the signaling network structure for the pathways leading to S-palmitoylation or any other such network being examined. Finally, it is also possible for the mutagenic element to integrate into a gene, even one critical for S-palmitoylation, without disrupting the function of the final translated protein. Due to the functional nature of the screen, a stable cell line with such a mutation would not be chosen for further analysis.

### EXAMPLE 10

### Identification and Functional Characterization of the Trapped Genes

Genes that when mutagenized will inhibit the process of S-palmitoylation are identified by the invention methods. It is also likely that many rounds of mutagenesis will be necessary to find candidate clonal lines as well as to understand the degree to which the genome has been saturated. 5′- and 3′-RACE methods (Frohman et al., 1988; Zambrowicz et al., 1997) can be used to identify trapped genes in cell lines that have the morphological hallmark of a mutagenized gene critical for S-palmitoylation. The vectors have been carefully designed so that unique sequence in SA, SD and GFP:SPS can be used in combination with universal primers and adaptors and protocols that are standard in the lab to rapidly and efficiently identify the locus of integration of the mutagenic reporters. Identification of the mutated gene by sequence analysis allows one to predict, in most cases, a possible function for the gene. However, further analysis may be desirable to understand the role of the identified genes in the pathway leading to S-palmitoylation. It is likely that genes involved in the synthesis of required precursors, as well as S-PATs, will be trapped. The background, false-positive clones are expected to outnumber the clones in which S-PATS are trapped with a significant number of the false positives occurring due to fragmentation of the reporter construct. As mentioned above, it is possible that the reporters could be physically fractured during the integration event (Voss et al., 1998) giving rise to independent, fractional integrations. Therefore, all clonal lines reporting a positive result are surveyed for expression of an un-fragmented reporter, GFP:SPS. Reverse transcription-PCR and/or PCR of genomic DNA using primers to the 5′ end of GFP and the 3′ end of the SPS are efficient ways to determine the integrity of the integrated reporter. This type of prescreening quickly reduces the potential positive clones to a workable number. The number of critical genes identified is expected to be in the tens, not hundreds or thousands. Among these, the most interesting will be singled out for more extensive characterization.

The polyA-trap-style of gene trap vector does not trap genes without introns. However, this smaller genomic space can be explored using promoter-trap vectors either as a supplement to the information that we gathered using polyA traps or as a backup in case we don't find critical genes searching with the polyA traps. While this genomic space is smaller, it is possible that the family(s) of genes responsible for S-palmitoylation could all fall into the intronless category.

### EXAMPLE 11

### Additional Methods to Characterize the Role of Trapped Genes in S-Palmitoylation Rescue of the Mutant Phenotype

After identifying a candidate, “critical”, trapped gene, a “rescue” of the mutant phenotype (i.e., GFP:SPS in the cytoplasm) is carried out by re-expressing the wild-type version of the mutated gene in the mutant cell line. This requires cloning the full-length cDNA of the mutated gene, putting it into a suitable expression vector such as pcDNA3 (Invitrogen) and reintroducing the gene into the mutant cell line. Successful rescue of the mutant phenotype, as observed by the GFP:SPS relocalizing back onto the PM, provides additional functional information supporting the identity of a gene involved in S-palmitoylation of the substrate used in the initial screen. An inability to rescue the mutant phenotype suggests that the full extent of mutagenic integrations was not properly characterized, at which point different mutagenized clones would be sought.

### EXAMPLE 12

Cross-Substrate Specificity Tests

It is possible that there are many S-PATs, each with unique substrate specificity. This hypothesis can be tested by expressing different, contextually unique S-palmitoylation substrates fused to another color of fluorescent protein (e.g., monomeric red fluorescent protein (mRFP) (Campbell et al., 2002)) in clonal cell lines containing putative or known S-PAT mutations. The red fluorescence of mRFP is easy to separate spectrally from GFP and gives simultaneous readouts of the subcellular localization of the two SPSs. Determining the degree of overlap among the other SPSs, and the original GFP:SPS provides important information aiding the prediction of the numbers of PATs and their specificity. Representatives from three of the four classes of SPSs as described in (Linder & Deschenes, 2003) can also be tested. Specifically, the C-terminal tail of CD151 (residues 236-end)(Yang et al., 2002); the c-terminus of Rho (Zacharias et al., 2002); The N-terminus of GAP-43 (Zacharias et al., 2002). The fourth class is mitochondrial proteins. The experiments described herein are not designed to determine the palmitoylation state of proteins localized to the mitochondria.

### EXAMPLE 13

Increasing the Morphological Homogeneity of the Reporter Cell Line

A potentially useful alternative approach that can be explored if necessary is to create a stable cell line constitutively expressing the GFP:SPS of choice and then performing gene-trapping on this cell line using G-418 resistance as the only marker for selection of mutagenic integrations. The phenotypic marker for integration of the construct into a gene relevant to S-palmitoylation would remain redistribution of the fluorescent marker from the PM to the cytoplasm. While this approach does not utilize the convenience of fluorescence as a marker for integration, preselecting a line stably, efficiently and correctly expressing the GFP:SPS could increase the ability to distinguish mutagenic events that are truly disruptive of proper S-palmitoylation.

Identification of the trapped genes by sequence analysis and database homology searches, combined with the direct tests of functionality described above allows one to reconstruct, at least in part, the genes that are required to induce S-palmitoylation of a protein. The identities of the genes make it possible to place them in a logical sequence that leads to S-palmitoylation.

In sum the preliminary data presented herein demonstrate:
1. Palmitoylation alone is sufficient to take the fluorescent reporter GFP to the plasma membrane.
2. The S-palmitoylation substrate, when fused to GFP, can report S-PAT activity independent of any other cellular activity.
3. The HTM component of this work does not represent an unachievable roadblock to successful completion of all of the goals set forth herein.

The invention has been described in detail with reference to preferred embodiments thereof However, it will be appreciated that those skilled in the art, upon consideration of this disclosure, may make modification and improvements within the spirit and scope of the invention as set forth in the following claims.

### Literature Cited:

Adams, S. R., Campbell, R. E., Gross, L. A., Martin, B. R., Walkup, G. K., Yao, Y., Llopis, J., and Tsien, R. Y. (2002) New biarsenical ligands and tetracysteine motifs for protein labeling in vitro and in vivo: synthesis and biological applications. J Am Chem Soc. 124(21):6063-76.
Bartels, D. J., Mitchell, D. A., Dong, X., and Deschenes, R. J. (1999) Erf2, a novel gene product that affects the localization and palmitoylation of Ras2 in Saccharomyces cerevisiae. Mol Cell Biol 19, 6775-87.
Berger, M., and Schmidt, M. F. (1984) Cell-free fatty acid acylation of Semliki Forest viral polypeptides with microsomal membranes from eukaryotic cells. J Biol Chem 259, 7245-52.
Berthiaume, L., and Resh, M. D. (1995) Biochemical characterization of a palmitoyl acyltransferase activity that palmitoylates myristoylated proteins. J Biol Chem 270, 22399-405.
Bharadwaj, M., and Bizzozero, O. A. (1995) Myelin P0 glycoprotein and a synthetic peptide containing the palmitoylation site are both autoacylated. J Neurochem 65, 1805-15.
Boland, M. V., Markey, M. K., and Murphy, R. F. (1998) Automated recognition of patterns characteristic of subcellular structures in fluorescence microscopy images. Cytometry 33, 366-75.
Boland, M. V., and Murphy, R. F. (1999a) After sequencing: quantitative analysis of protein localization. IEEE Eng Med Biol Mag 18, 115-9.
Boland, M. V., and Murphy, R. F. (1999b) Automated analysis of patterns in fluorescence-microscope images. Trends Cell Biol 9, 201-2.
Boland, M. V., and Murphy, R. F. (2001) A neural network classifier capable of recognizing the patterns of all major subcellular structures in fluorescence microscope images of HeLa cells. Bioinformatics 17, 1213-23.
Brigger, P., Muller, F., Illgner, K., and Unser, M. (1999) Centered Pyramids. IEEE Transactions on Image Processing, 8, 1254-1264.
Brigger, P., Hoeg, J., and Unser, M. (2000) B-Spline Snakes: A flexible Tool for Parametric Contour Detection. IEEE Transactions on Image Processing, 9, 1484-1496.
Camp, L. A., and Hofinann, S. L. (1993) Purification and properties of a palmitoyl-protein thioesterase that cleaves palmitate from H-Ras. J Biol Chem 268, 22566-74.
Camp, L. A., Verkruyse, L. A., Afendis, S. J., Slaughter, C. A., and Hofmann, S. L. (1994) Molecular cloning and expression of palmitoyl-protein thioesterase. J Biol Chem 269, 23212-9.
Campbell, R. E., Tour, O., Palmer, A. E., Steinbach, P. A., Baird, G. S., Zacharias, D. A., and Tsien, R. Y. (2002) A monomeric red fluorescent protein. Proc Natl Acad Sci U S A 99, 7877-82.
Canny, J. (1986) A Compuntational Approach to Edge Detection. IEEE Transactions on Pattern Analysis and Machine Intelligence 8, 679-697.
Cecconi, F., and Meyer, B. I. (2000) Gene trap: a way to identify novel genes and unravel their biological function. FEBS Lett 480, 63-71.
Cecconi, F., and Gruss, P. (2002) From ES cells to mice: the gene trap approach. Methods Mol Biol 185, 335-46.
Chowdhury, K., Bonaldo, P., Torres, M., Stoykova, A., and Gruss, P. (1997) Evidence for the stochastic integration of gene trap vectors into the mouse germline. Nucleic Acids Res 25, 1531-6.
Conway, B. R., Minor, L. K., Xu, J. Z., D'Andrea, M. R., Ghosh, R. N., and Demarest, K. T. (2001) Quantitative analysis of agonist-dependent parathyroid hormone receptor trafficking in whole cells using a functional green fluorescent protein conjugate. J Cell Physiol 189, 341-55.
Craven, S. E., El-Husseini, A. E., and Bredt, D. S. (1999) Synaptic targeting of the postsynaptic density protein PSD-95 mediated by lipid and protein motifs. Neuron 22, 497-509.
Davies, E. (1997) Machine Vision: Theory, Algorithms, Practicalities, 2nd ed., Academic Press.
Ding, G. J., Fischer, P. A., Boltz, R. C., Schmidt, J. A., Colaianne, J. J., Gough, A., Rubin, R. A., and Miller, D. K. (1998) Characterization and quantitation of NF-kappaB nuclear translocation induced by interleukin-1 and tumor necrosis factor-alpha. Development and use of a high capacity fluorescence cytometric system. J Biol Chem 273, 28897-905.
Dinsmore, C. J., and Bell, I. M. (2003) Inhibitors of farnesyltransferase and geranylgeranyltransferase-1 for antitumor therapy: substrate-based design, conformational constraint and biological activity. Curr Top Med Chem 3, 1075-93.
Dong, X., Mitchell, D. A., Lobo, S., Zhao, L., Bartels, D. J., and Deschenes, R. J. (2003) Palmitoylation and Plasma Membrane Localization of Ras2p by a Nonclassical Trafficking Pathway in Saccharomyces cerevisiae. Mol Cell Biol 23, 6574-84.
Duda, R., and Hart, P. (1972) Use of Hough Transform to Detect Lines and Curves in Pictures. Communications of the ACM 15, 11-15.
Dudler, T., and Gelb, M. H. (1996) Palmitoylation of Ha-Ras facilitates membrane binding, activation of downstream effectors, and meiotic maturation in Xenopus oocytes. J Biol Chem 271, 11541-7.
Duncan, J. A., and Gilman, A. G. (1996) Autoacylation of G protein alpha subunits. J Biol Chem 271, 23594-600.
Duncan, J. A., and Gilman, A. G. (1998) A cytoplasmic acyl-protein thioesterase that removes palmitate from G protein alpha subunits and p21 (RAS). J Biol Chem 273, 15830-7.
Dunphy, J. T., Greentree, W. K., Manahan, C. L., and Linder, M. E. (1996) G-protein palmitoyltransferase activity is enriched in plasma membranes. J Biol Chem 271, 7154-9.
El-Husseini, A. E., Craven, S. E., Chetkovich, D. M., Firestein, B. L., Schnell, E., Aoki, C., and Bredt, D. S. (2000) Dual palmitoylation of PSD-95 mediates its vesiculotubular sorting, postsynaptic targeting, and ion channel clustering. J Cell Biol 148, 159-72.
El-Husseini, A. E., Topinka, J. R., Lehrer-Graiwer, J. E., Firestein, B. L., Craven, S. E., Aoki, C., and Bredt, D. S. (2000) Ion channel clustering by membrane-associated guanylate kinases. Differential regulation by N-terminal lipid and metal binding motifs. J Biol Chem 275, 23904-10.
el-Husseini Ael, D., and Bredt, D. S. (2002) Protein palmitoylation: a regulator of neuronal development and function. Nat Rev Neurosci 3, 791-802.
El-Husseini Ael, D., Schnell, E., Dakoji, S., Sweeney, N., Zhou, Q., Prange, O., Gauthier-Campbell, C., Aguilera-Moreno, A., Nicoll, R. A., and Bredt, D. S. (2002) Synaptic strength regulated by palmitate cycling on PSD-95. Cell 108, 849-63.
Emi, N., Friedmann, T., and Yee, J. K. (1991) Pseudotype formation of murine leukemia virus with the G protein of vesicular stomatitis virus. J Virol 65, 1202-7.
Evans, M. J., Carlton, M. B., and Russ, A. P. (1997) Gene trapping and functional genomics. Trends Genet 13, 370-4.
Evans, M. J. (1998) Gene trapping—a preface. Dev Dyn 212, 167-9.
Eviatar, H., and Somorjai, R. (1996) A Fast Simple Active Contour Algorithm for Biomedical Images. Pattern Recognition Letters 17, 969-974.
Farazi, T. A., Waksman, G., and Gordon, J. I. (2001) The biology and enzymology of protein N-myristoylation. J Biol Chem 276, 39501-4.
Forsyth, D. a. P., J (2003) Computer Vision: A Modern Approach, Prentice Hall.
Friedrich, G., and Soriano, P. (1991) Promoter traps in embryonic stem cells: a genetic screen to identify and mutate developmental genes in mice. Genes Dev 5, 1513-23.
Frohman, M. A., Dush, M. K., and Martin, G. R. (1988) Rapid production of full-length cDNAs from rare transcripts: amplification using a single gene-specific oligonucleotide primer. Proc Natl Acad Sci U S A 85, 8998-9002.
Fu, H. W., and Casey, P. J. (1999) Enzymology and biology of CaaX protein prenylation. Recent Prog Horm Res 54, 315-42; discussion 342-3.
Gentles, A. J., and Karlin, S. (1999) Why are human G-protein-coupled receptors predominantly intronless? Trends Genet 15, 47-9.
Ghobrial, I. M., and Adjei, A. A. (2002) Inhibitors of the ras oncogene as therapeutic targets. Hematol Oncol Clin North Am 16, 1065-88.
Ghosh, R. N., Chen, Y. T., DeBiasio, R., DeBiasio, R. L., Conway, B. R., Minor, L. K., and Demarest, K. T. (2000) Cell-based, high-content screen for receptor internalization, recycling and intracellular trafficking. Biotechniques 29, 170-5.
Giuliano, K. A., and Taylor, D. L. (1998) Fluorescent-protein biosensors: new tools for drug discovery. Trends Biotechnol 16, 135-40.
Gupta, P., Soyombo, A. A., Atashband, A., Wisniewski, K. E., Shelton, J. M., Richardson, J. A., Hammer, R. E., and Hofmann, S. L. (2001) Disruption of PPT1 or PPT2 causes neuronal ceroid lipofuscinosis in knockout mice. Proc Natl Acad Sci U S A 98, 13566-71.
Griffin, B. A., Adams, S. R., Jones, J., and Tsien, R. Y. (2000) Fluorescent labeling of recombinant proteins in living cells with FlAsH. Methods Enzymol. 327:565-78.
Hancock, J. F., Paterson, H., and Marshall, C. J. (1990) A polybasic domain or palmitoylation is required in addition to the CAAX motif to localize p21ras to the plasma membrane. Cell 63, 133-9.
Hess, D. T., Patterson, S. I., Smith, D. S., and Skene, J. H. (1993) Neuronal growth cone collapse and inhibition of protein fatty acylation by nitric oxide. Nature 366, 562-5.
Hicks, G. G., Shi, E. G., Li, X. M., Li, C. H., Pawlak, M., and Ruley, H. E. (1997) Functional genomics in mice by tagged sequence mutagenesis. Nat Genet 16, 338-44.
Hiol, A., Davey, P. C., Osterhout, J. L., Waheed, A. A., Fischer, E. R., Chen, C. K., Milligan, G., Druey, K. M., and Jones, T. L. (2003) Palmitoylation regulates regulators of G-protein signaling (RGS) 16 function. I. Mutation of amino-terminal cysteine residues on RGS 16 prevents its targeting to lipid rafts and palmitoylation of an internal cysteine residue. J Biol Chem 278, 19301-8.
Hofemeister, H., Kuhn, C., Franke, W. W., Weber, K., and Stick, R. (2002) Conservation of the gene structure and membrane-targeting signals of germ cell-specific lamin LIII in amphibians and fish. Eur J Cell Biol 81, 51-60.
Illingworth, J., and Kittler, J. (1988) A Survey of the Hough Transform. Computer Vision, Graphics and Image Processing 44.
Kanaani, J., el-Husseini Ael, D., Aguilera-Moreno, A., Diacovo, J. M., Bredt, D. S., and Baekkeskov, S. (2002) A combination of three distinct trafficking signals mediates axonal targeting and presynaptic clustering of GAD65. J Cell Biol 158, 1229-38.
Kass, M., Witkin, A., and Terzopoulos, D. (1988) Snakes: Active Countour Models. International Journal of Computer Vision, 321-331.
Klein, A., Egglin, T., Pollak, J., Lee, F., and Amini, A. (1994) Identifying Vascular Features with Orientation-Specific Filters adn B-Spline Snakes. Computational Cardiology, 113-116.
Kleuss, C., and Krause, E. (2003) Galpha(s) is palmitoylated at the N-terminal glycine. Embo J 22, 826-32.
Lane, S. R., and Liu, Y. (1997) Characterization of the palmitoylation domain of SNAP-25. J Neurochem 69, 1864-9.
Linder, M. E., and Deschenes, R. J. (2003) New insights into the mechanisms of protein palmitoylation. Biochemistry 42, 4311-20.
Lippincott-Schwartz, J., and Patterson, G. H. (2003) Development and use of fluorescent protein markers in living cells. Science 300, 87-91.
Liu, H., Abecasis, G. R., Heath, S. C., Knowles, A., Demars, S., Chen, Y. J., Roos, J. L., Rapoport, J. L., Gogos, J. A., and Karayiorgou, M. (2002) Genetic variation in the 22q11 locus and susceptibility to schizophrenia. Proc Natl Acad Sci U S A 99, 16859-64.
Lobregt, S., and Liergever, M. (1995) A Discrete Dynamic Contour Model. IEEE Transactions on medical Imaging 14, 12-24.
Long, S. B., Casey, P. J., and Beese, L. S. (2002) Reaction path of protein famesyltransferase at atomic resolution. Nature 419, 645-50.
Loranger, S. S., and Linder, M. E. (2002) SNAP-25 traffics to the plasma membrane by a syntaxin-independent mechanism. J Biol Chem 277, 34303-9.
Luebke, K. J. (1998) A FLASH of insight into cellular chemistry: genetically encoded labels for protein visualization in vivo. Chem. Biol. 5(12):R317-22.
Mack, D., Berger, M., Schmidt, M. F., and Kruppa, J. (1987) Cell-free fatty acylation of microsomal integrated and detergent-solubilized glycoprotein of vesicular stomatitis virus. J Biol Chem 262, 4297-302.
Marek, K. W. and Davis, G. W. (2002) Transgenically encoded protein photoinactivation (FlAsH-FALI): acute inactivation of synaptotagmin I. Neuron. 36(5):805-13.
Markey, M. K., Boland, M. V., and Murphy, R. F. (1999) Toward objective selection of representative microscope images. Biophys J 76, 2230-7.
Marr, D. (1982) Vision, W. H. Freeman and Company.
Mcinerney, T., and Terzopoulos, D. (1995) in 5th International Conference on Computer Vision pp 840-845.
Melkonian, K. A., Ostermeyer, A. G., Chen, J. Z., Roth, M. G., and Brown, D. A. (1999) Role of lipid modifications in targeting proteins to detergent-resistant membrane rafts. Many raft proteins are acylated, while few are prenylated. J Biol Chem 274, 3910-7.
Menet, S., Saint-Marc, P., and Melioni, G. (1990) in Image Understanding Workshop pp 720-726, Morgan Kaufman.
Milligan, G. (2003) High-content assays for ligand regulation of G-protein-coupled receptors. Drug Discov Today 8, 579-85.
Minguez, J. M., Giuliano, K. A., Balachandran, R., Madiraju, C., Curran, D. P., and Day, B. W. (2002) Synthesis and high content cell-based profiling of simplified analogues of the microtubule stabilizer (+)-discodermolide. Mol Cancer Ther 1, 1305-13.
Moffett, S., Brown, D. A., and Linder, M. E. (2000) Lipid-dependent targeting of G proteins into rafts. J Biol Chem 275, 2191-8.
Murphy, R. F., Boland, M. V., and Velliste, M. (2000) Towards a systematics for protein subcelluar location: quantitative description of protein localization patterns and automated analysis of fluorescence microscope images. Proc Int Conf Intell Syst Mol Biol 8, 251-9.
Niwa, H., Araki, K., Kimura, S., Taniguchi, S., Wakasugi, S., and Yamamura, K. (1993) An efficient gene-trap method using poly A trap vectors and characterization of gene-trap events. J Biochem (Tokyo) 113, 343-9.
Osterhout, J. L., Waheed, A. A., Hiol, A., Ward, R. J., Davey, P. C., Nini, L., Wang, J., Milligan, G., Jones, T. L., and Druey, K. M. (2003) Palmitoylation regulates regulator of G-protein signaling (RGS) 16 function. II. Palmitoylation of a cysteine residue in the RGS box is critical for RGS 16 GTPase accelerating activity and regulation of Gi-coupled signalling. J Biol Chem 278, 19309-16.
Ovchinnikov Yu, A., Abdulaev, N. G., and Bogachuk, A. S. (1988) Two adjacent cysteine residues in the C-terminal cytoplasmic fragment of bovine rhodopsin are palmitylated. FEBS Lett 230, 1-5.
Pao, D., and Li, H. (1992) Shapes Recognition Using the Straight Line Hough Transform: Theory and Generalization. IEEE Transactions on Pattern Analysis and Machine Intelligence 14, 1076-1089.
Park, H., Hanson, G. T., Duff, S. R., and Selvin, P. R. (2004) Nanometre localization of single ReAsH molecules. J. Microsc. 216(pt 3):199-205.
Pepinsky, R. B., Zeng, C., Wen, D., Rayhom, P., Baker, D. P., Williams, K. P., Bixler, S. A., Ambrose, C. M., Garber, E. A., Miatkowski, K., Taylor, F. R., Wang, E. A., and Galdes, A. (1998) Identification of a palmitic acid-modified form of human Sonic hedgehog. J Biol Chem 273, 14037-45.
Perez, A. S., and Bredt, D. S. (1998) The N-terminal PDZ-containing region of postsynaptic density-95 mediates association with caveolar-like lipid domains. Neurosci Lett 258, 121-3.
Piegl, L. T., W (1997) The NURBS Book, 2nd ed., Springer-Verlag.
Price, J. H., Hunter, E. A., and Gough, D. A. (1996) Accuracy of least squares designed spatial FIR filters for segmentation of images of fluorescence stained cell nuclei. Cytometry 25, 303-16.
Price, J. H., Goodacre, A., Hahn, K., Hodgson, L., Hunter, E. A., Krajewski, S., Murphy, R. F., Rabinovich, A., Reed, J. C., and Heynen, S. (2002) Advances in molecular labeling, high throughput imaging and machine intelligence portend powerful functional cellular biochemistry tools. J Cell Biochem Suppl 39, 194-210.
Prior, I. A., and Hancock, J. F. (2001) Compartmentalization of Ras proteins. J Cell Sci 114, 1603-8.
Putilina, T., Wong, P., and Gentleman, S. (1999) The DHHC domain: a new highly conserved cysteine-rich motif. Mol Cell Biochem 195, 219-26.
Qanbar, R., and Bouvier, M. (2003) Role of palmitoylation/depalmitoylation reactions in G-protein-coupled receptor function. Pharmacol Ther 97, 1-33.
Quesnel, S., and Silvius, J. R. (1994) Cysteine-containing peptide sequences exhibit facile uncatalyzed transacylation and acyl-CoA-dependent acylation at the lipid bilayer interface. Biochemistry 33, 13340-8.
Rajala, R. V., Datla, R. S., Moyana, T. N., Kakkar, R., Carlsen, S. A., and Sharma, R. K. (2000) N-myristoyltransferase. Mol Cell Biochem 204, 135-55.
Ranganath, S. (1995) Contour Extraction from Cardiac MRI Studies Using Snakes. IEEE Transactions on Medical Imaging 14, 328-338.
Reddy, B. A., Kloc, M., and Etkin, L. D. (1991) Identification of the cDNA for xlcaax-1, a membrane associated Xenopus maternal protein. Biochem Biophys Res Commun 179, 1635-41.
Resh, M. D. (1999) Fatty acylation of proteins: new insights into membrane targeting of myristoylated and palmitoylated proteins. Biochim Biophys Acta 1451, 1-16.
Roberts, R. M., Liu, L., Guo, Q., Leaman, D., and Bixby, J. (1998) The evolution of the type I interferons. J Interferon Cytokine Res 18, 805-16.
Robinson, L. C., Bradley, C., Bryan, J. D., Jerome, A., Kweon, Y., and Panek, H. R. (1999) The Yck2 yeast casein kinase 1 isoform shows cell cycle-specific localization to sites of polarized growth and is required for proper septin organization. Mol Biol Cell 10, 1077-92.
Roques, E. J., and Murphy, R. F. (2002) Objective evaluation of differences in protein subcellular distribution. Traffic 3, 61-5.
Rosenfeld, A. (1984) Multiresolution Image Processing, Springer-Verlag.
Roskoski, R., Jr. (2003) Protein prenylation: a pivotal posttranslational process. Biochem Biophys Res Commun 303, 1-7.
Roth, A. F., Feng, Y., Chen, L., and Davis, N. G. (2002) The yeast DHHC cysteine-rich domain protein Akrlp is a palmitoyl transferase. J Cell Biol 159, 23-8.
Sadeghi, H. M., Innamorati, G., Dagarag, M., and Bimbaumer, M. (1997) Palmitoylation of the V2 vasopressin receptor. Mol Pharmacol 52, 21-9.
Salminen, M., Meyer, B. I., and Gruss, P. (1998) Efficient poly A trap approach allows the capture of genes specifically active in differentiated embryonic stem cells and in mouse embryos. Dev Dyn 212, 326-33.
Sinensky, M. (2000) Recent advances in the study of prenylated proteins. Biochim Biophys Acta 1484, 93-106.
Sosinsky, A., Glusman, G., and Lancet, D. (2000) The genomic structure of human olfactory receptor genes. Genomics 70, 49-61.
Stanford, W. L., Cohn, J. B., and Cordes, S. P. (2001) Gene-trap mutagenesis: past, present and beyond. Nat Rev Genet 2, 756-68.
Stoffel, R. H., Randall, R. R., Premont, R. T., Lefkowitz, R. J., and Inglese, J. (1994) Palmitoylation of G protein-coupled receptor kinase, GRK6. Lipid modification diversity in the GRK family. J Biol Chem 269, 27791-4.
Taylor, D. L., Woo, E. S., and Giuliano, K. A. (2001) Real-time molecular and cellular analysis: the new frontier of drug discovery. Curr Opin Biotechnol 12, 75-81.
Topinka, J. R., and Bredt, D. S. (1998) N-terminal palmitoylation of PSD-95 regulates association with cell membranes and interaction with K+channel Kv1.4. Neuron 20, 125-34.
Tour, O., Meijer, R. M., Zacharias, D. A., Adams, S. R., and Tsien, R. Y. (2003) Genetically targeted chromophore-assisted light inactivation. Nat. Biotechnol. 21(12):1505-8.
Vesa, J., Hellsten, E., Verkruyse, L. A., Camp, L. A., Rapola, J., Santavuori, P., Hofinann, S. L., and Peltonen, L. (1995) Mutations in the palmitoyl protein thioesterase gene causing infantile neuronal ceroid lipofuscinosis. Nature 376, 584-7.
Voss, A. K., Thomas, T., and Gruss, P. (1998) Efficiency assessment of the gene trap approach. Dev Dyn 212, 171-80.
Wang, M., Evans, J., Hassebrook, L., and Knapp, C. (1996) A Multistage, Optimal Active Countour Model. IEEE Transactions on Image Processing, 5, 1586-1591.
Weijer, C. J. (2003) Visualizing signals moving in cells. Science 300, 96-100.
Wiedmer, T., Zhao, J., Nanjundan, M., and Sims, P. J. (2003) Palmitoylation of phospholipid scramblase 1 controls its distribution between nucleus and plasma membrane. Biochemistry 42, 1227-33.
Wolven, A., Okamura, H., Rosenblatt, Y., and Resh, M. D. (1997) Palmitoylation of p59fyn is reversible and sufficient for plasma membrane association. Mol Biol Cell 8, 1159-73.
Woollacott, A. J., and Simpson, P. B. (2001) High throughput fluorescence assays for the measurement of mitochondrial activity in intact human neuroblastoma cells. J Biomol Screen 6, 413-20.
Wurst, W., and Joyner, A. L. (1993) Gene Targeting: A Practical Approach, Oxford University Press, Oxford, UK.
Yang, M., Lee, J., Lien, C., and Huang, C. (1997) Hough Transform Modified by Line Connectivity and Line Thickness. IEEE Transactions on Pattern Analysis and Machine Intelligence 19, 905-910.
Yang, X., Claas, C., Kraeft, S. K., Chen, L. B., Wang, Z., Kreidberg, J. A., and Hemler, M. E. (2002) Palmitoylation of tetraspanin proteins: modulation of CD151 lateral interactions, subcellular distribution, and integrin-dependent cell morphology. Mol Biol Cell 13, 767-81.
Yarrow, J. C., Feng, Y., Perlman, Z. E., Kirchhausen, T., and Mitchison, T. J. (2003) Phenotypic screening of small molecule libraries by high throughput cell imaging. Comb Chem High Throughput Screen 6, 279-86.
Zacharias, D. A., Baird, G. S., and Tsien, R. Y. (2000) Recent advances in technology for measuring and manipulating cell signals. Curr Opin Neurobiol 10, 416-21.
Zacharias, D. A., Violin, J. D., Newton, A. C., and Tsien, R. Y. (2002) Partitioning of lipid-modified monomeric GFPs into membrane microdomains of live cells. Science 296, 913-6.
Zambrowicz, B. P., Imamoto, A., Fiering, S., Herzenberg, L. A., Kerr, W. G., and Soriano, P. (1997) Disruption of overlapping transcripts in the ROSA beta geo 26 gene trap strain leads to widespread expression of beta-galactosidase in mouse embryos and hematopoietic cells. Proc Natl Acad Sci U S A 94, 3789-94.
Zambrowicz, B. P., Friedrich, G. A., Buxton, E. C., Lilleberg, S. L., Person, C., and Sands, A. T. (1998) Disruption and sequence identification of 2,000 genes in mouse embryonic stem cells. Nature 392, 608-11.

What is claimed is:

1. A method for identifying a gene that modulates subcellular localization of a protein, said method comprising:
a) contacting a haploid or hypodiploid cell which expresses said protein with an insertional mutagen under conditions suitable to produce modified haploid or hypodiploid cell(s),
b) detecting a change in the subcellular localization of the protein as a result of the insertional mutagen; and
c) identifying, in those modified haploid or hypodiploid cells in which a change in the subcellular localization of the protein is detected, the gene into which the insertional mutagen is inserted, thereby identifying the gene that modulates subcellular localization of the protein.

2. A method for identifying a gene that modulates subcellular localization of a protein, said method comprising:
a) contacting a haploid or hypodiploid cell which expresses said protein with an insertional mutagen under conditions suitable to produce modified haploid or hypodiploid cell(s),
b) selecting those modified haploid or hypodiploid cells which reveal a change in the subcellular localization of the protein; and
c) identifying, in the modified haploid or hypodiploid cells selected in step (b), the gene into which the insertional mutagen is inserted, thereby identifying the gene that modulates subcellular localization of the protein.

3. A method for identifying a gene that modulates subcellular localization of a protein, said method comprising:
a) selecting modified haploid or hypodiploid cell(s) which express said protein and which reveal a change in the subcellular localization of the protein upon contacting with an insertional mutagen, and
b) identifying, in the modified haploid or hypodiploid cells selected in step (a), the gene into which the insertional mutagen is inserted, thereby identifying the gene that modulates subcellular localization of the protein.

4. A method for identifying a gene that modulates subcellular localization of a protein, said method comprising:
a) contacting a haploid or hypodiploid cell which expresses said protein with an insertional mutagen under conditions suitable to produce modified haploid or hypodiploid cell(s); and
b) identifying, in those modified haploid or hypodiploid cells in which a change in the subcellular localization of the protein occurs, the gene into which the insertional mutagen is inserted, thereby identifying the gene that modulates subcellular localization of the protein.

5. A method for identifying a gene that modulates subcellular localization of a protein, said method comprising identifying, in those modified haploid or hypodiploid cells which reveal a change in the subcellular localization of the protein upon introduction of an insertional mutagen thereto, the gene into which the insertional mutagen is inserted, thereby identifying the gene that modulates subcellular localization of the protein.

6. A method for identifying a gene that modulates cell morphology, said method comprising identifying, in those modified haploid or hypodiploid cells which reveal a change in the cell morphology upon introduction of an insertional mutagen thereto, the gene into which the insertional mutagen is inserted, thereby identifying the gene that modulates cell morphology.

7. A method for determining the enzymatic cascade of genes responsible for a protein modification of interest, said method comprising:
randomly mutagenizing a haploid or hypodiploid cell, optionally containing one or more stably expressed marker gene(s), with a mutagenic element,
selecting/detecting those cell lines that harbor the mutagenic element, and optionally one or more stably expressed marker gene(s), and
identifying, in the modified haploid or hypodiploid cells selected/detected above, a change in phenotype, thereby identifying a gene in the enzymatic cascade of interest.

8. A method for determining the enzymatic cascade of genes responsible for a protein modification of interest, said method comprising:
selecting/detecting those haploid or hypodiploid cells, optionally containing one or more stably expressed marker gene(s), that harbor a mutagenic element, and optionally one or more stably expressed marker gene(s) as a result of being randomly mutagenized with a mutagenic element, and
identifying, in the modified haploid or hypodiploid cells selected/detected above, a change in phenotype, thereby identifying a gene in the enzymatic cascade of interest.

9. A method for determining the enzymatic cascade of genes responsible for a protein modification of interest, said method comprising identifying a change in phenotype in modified haploid or hypodiploid cells prepared by randomly mutagenizing a haploid or hypodiploid cell, optionally containing one or more stably expressed marker gene(s), with a mutagenic element, thereby identifying a gene in the enzymatic cascade of interest.

10. A stable, haploid or hypodiploid line expressing a detectable marker, operably associated with a substrate for a reaction of interest.

11. A line according to claim 10 wherein said detectable marker is a fluorophore, a chromophore or a chromogenic substrate.

12. A line according to claim 10 wherein said detectable marker is a fluorophore.

13. A line according to claim 12 wherein said fluorophore is Green Fluorescent Protein (GFP).

14. A line according to claim 10 wherein said reaction of interest is palmitoylation.

15. A line according to claim 14 wherein the substrate is a short peptide.

16. A line according to claim 15 wherein said short peptide substrate is an S-palmitoylation substrate.

17. A line according to claim 10 wherein said reaction of interest is prenylation.

18. A line according to claim 10 wherein said reaction of interest is acylation.

19. A line according to claim 10 wherein said reaction of interest is regulation of transcription, the action of steroids and steroid-like compounds, neuroregeneration and spinal cord repair, cell cycle regulation, stem cells and neuronal stem cells, cell migration, filopodia, GPCR-related signaling, phosphorylation, contact-inhibition of cell growth, tumorigenesis, identification of the molecular targets of drugs with unknown mechanism(s) of action, and any signaling pathway or cellular process in which a unique morphological metric can be tied (either directly or indirectly) to that process.
